SEQUENCE LISTING (5 sequences)

SEQ ID NO: 1 - protein, 511 aa, Artificial Sequence
  amino acids sequence encoding SUMO-GOT1 fusion protein
MGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGAPPSVFAEVPQAQPVLVFKLTADFREDPDPRKVNLGVGAYRTDDCHPWVLPVVKKVEQKIANDNSLNHEYLPILGLAEFRSCASRLALGDDSPALKEKRVGGVQSLGGTGALRIGADFLARWYNGTNNKNTPVYVSSPTWENHNAVFSAAGFKDIRSYRYWDAEKRGLDLQGFLNDLENAPEFSIVVLHACAHNPTGIDPTPEQWKQIASVMKHRFLFPFFDSAYQGFASGNLERDAWAIRYFVSEGFEFFCAQSFSKNFGLYNERVGNLTVVGKEPESILQVLSQMEKIVRITWSNPPAQGARIVASTLSNPELFEEWTGNVKTMADRILTMRSELRARLEALKTPGTWNHITDQIGMFSFTGLNPKQVEYLVNEKHIYLLPSGRINVSGLTTKNLDYVATSIHEAVTKIQ

SEQ ID NO: 2 - protein, 412 aa, Artificial Sequence
  amino acids sequence encoding GOT1
APPSVFAEVPQAQPVLVFKLTADFREDPDPRKVNLGVGAYRTDDCHPWVLPVVKKVEQKIANDNSLNHEYLPILGLAEFRSCASRLALGDDSPALKEKRVGGVQSLGGTGALRIGADFLARWYNGTNNKNTPVYVSSPTWENHNAVFSAAGFKDIRSYRYWDAEKRGLDLQGFLNDLENAPEFSIVVLHACAHNPTGIDPTPEQWKQIASVMKHRFLFPFFDSAYQGFASGNLERDAWAIRYFVSEGFEFFCAQSFSKNFGLYNERVGNLTVVGKEPESILQVLSQMEKIVRITWSNPPAQGARIVASTLSNPELFEEWTGNVKTMADRILTMRSELRARLEALKTPGTWNHITDQIGMFSFTGLNPKQVEYLVNEKHIYLLPSGRINVSGLTTKNLDYVATSIHEAVTKIQ

SEQ ID NO: 3 - DNA, 1530 bp, Artificial Sequence
  nucleic acid sequence encoding SUMO-GOT1 fusion protein
ggatcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggtgcacctccgtcagtctttgccgaggttccgcaggcccagcctgtcctggtcttcaagctcactgccgacttcagggaggatccggacccccgcaaggtcaacctgggagtgggagcatatcgcacggatgactgccatccctgggttttgccagtagtgaagaaagtggagcagaagattgctaatgacaatagcctaaatcacgagtatctgccaatcctgggcctggctgagttccggagctgtgcttctcgtcttgcccttggggatgacagcccagcactcaaggagaagcgggtaggaggtgtgcaatctttggggggaacaggtgcacttcgaattggagctgatttcttagcgcgttggtacaatggaacaaacaacaagaacacacctgtctatgtgtcctcaccaacctgggagaatcacaatgctgtgttttccgctgctggttttaaagacattcggtcctatcgctactgggatgcagagaagagaggattggacctccagggcttcctgaatgatctggagaatgctcctgagttctccattgttgtcctccacgcctgtgcacacaacccaactgggattgacccaactccggagcagtggaagcagattgcttctgtcatgaagcaccggtttctgttccccttctttgactcagcctatcagggcttcgcatctggaaacctggagagagatgcctgggccattcgctattttgtgtctgaaggcttcgagttcttctgtgcccagtccttctccaagaacttcgggctctacaatgagagagtcgggaatctgactgtggttggaaaagaacctgagagcatcctgcaagtcctttcccagatggagaagatcgtgcggattacttggtccaatccccccgcccagggagcacgaattgtggccagcaccctctctaaccctgagctctttgaggaatggacaggtaatgtgaagacaatggctgaccggattctgaccatgagatctgaactcagggcacgactagaagccctcaaaacccctgggacctggaaccacatcactgatcaaattggcatgttcagcttcactgggttgaaccccaagcaggttgagtatctggtcaatgaaaagcacatctacctgctgccaagtggtcgaatcaacgtgagtggcttaaccaccaaaaatctagattacgtggccacctccatccatgaagcagtcaccaaaatccag

SEQ ID NO: 4 - DNA, 1239 bp, Artificial Sequence
  nucleic acid sequence encoding GOT1 protein
atggcacctccgtcagtctttgccgaggttccgcaggcccagcctgtcctggtcttcaagctcactgccgacttcagggaggatccggacccccgcaaggtcaacctgggagtgggagcatatcgcacggatgactgccatccctgggttttgccagtagtgaagaaagtggagcagaagattgctaatgacaatagcctaaatcacgagtatctgccaatcctgggcctggctgagttccggagctgtgcttctcgtcttgcccttggggatgacagcccagcactcaaggagaagcgggtaggaggtgtgcaatctttggggggaacaggtgcacttcgaattggagctgatttcttagcgcgttggtacaatggaacaaacaacaagaacacacctgtctatgtgtcctcaccaacctgggagaatcacaatgctgtgttttccgctgctggttttaaagacattcggtcctatcgctactgggatgcagagaagagaggattggacctccagggcttcctgaatgatctggagaatgctcctgagttctccattgttgtcctccacgcctgtgcacacaacccaactgggattgacccaactccggagcagtggaagcagattgcttctgtcatgaagcaccggtttctgttccccttctttgactcagcctatcagggcttcgcatctggaaacctggagagagatgcctgggccattcgctattttgtgtctgaaggcttcgagttcttctgtgcccagtccttctccaagaacttcgggctctacaatgagagagtcgggaatctgactgtggttggaaaagaacctgagagcatcctgcaagtcctttcccagatggagaagatcgtgcggattacttggtccaatccccccgcccagggagcacgaattgtggccagcaccctctctaaccctgagctctttgaggaatggacaggtaatgtgaagacaatggctgaccggattctgaccatgagatctgaactcagggcacgactagaagccctcaaaacccctgggacctggaaccacatcactgatcaaattggcatgttcagcttcactgggttgaaccccaagcaggttgagtatctggtcaatgaaaagcacatctacctgctgccaagtggtcgaatcaacgtgagtggcttaaccaccaaaaatctagattacgtggccacctccatccatgaagcagtcaccaaaatccag

SEQ ID NO: 5 - protein, 99 aa, Artificial Sequence
  amino acids sequence encoding SUMO
MGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

(19) United States
(12) Patent Application Publication
(10) Pub. No.: US 2018/0071369 A1
(43) Pub. Date: Mar. 15, 2018

(54) GLUTAMATE OXALOACETATE TRANSAMINASE 1 (GOT1), PREPARATIONS AND METHODS OF GENERATING SAME AND USES THEREOF

(71) Applicant: Yeda Research and Development Co. Ltd., Rehovot (IL)

(72) Inventors: David MIRELMAN, Ramat-Efal (IL); Aharon RABINKOV, Rehovot (IL); Angela RUBAN, Rehovot (IL); Ghil JONA, Rehovot (IL); Eli HAZUM, Rehovot (IL); Vladas BUMELIS, Rehovot (IL); Nerijus MAKAUSKAS, Rehovot (IL); Saule SUDZIUVIENE, Rehovot (IL); Elena NARMONTAITE, Rehovot (IL)

(21) Appl. No.: 15/561,053

(22) PCT Filed: Mar. 31, 2016
(86) PCT No.: PCT/IL2016/050346

§ 371 Date: Sep. 24, 2017

Related U.S. Application Data
(60) Provisional application No. 62/140,492, filed on Mar. 31, 2015.

(51) Int. Cl. A61K 38/45; C12N 9/10

(52) U.S. Cl. CPC A61K 38/45; C12Y 206/01001; C12N 9/1096

(57) ABSTRACT

A protein preparation comprising Glutamate Oxaloacetate Transaminase 1 (GOT1) polypeptide molecules is disclosed, the GOT1 being identical in its sequence to that present in human serum. 100% of the GOT1 polypeptide molecules of the preparation have an alanine at position 1 of the GOT1 polypeptide. The GOT1 polypeptide molecules constitute at least 95% of the proteins in the preparation. Methods of generating same and uses thereof are also disclosed.

### FIELD AND BACKGROUND OF THE INVENTION

The present invention, in some embodiments thereof, relates to purified Glutamate Oxaloacetate Transaminase 1 (GOT1) preparations and to methods of generating same.

Glutamate Oxaloacetate Transaminase 1 (GOT1) is a serum enzyme which regulates the blood levels of glutamate by converting it to alfa-keto glutarate. In animal models the systemic administration of recombinant Glutamate Oxaloacetate Transaminase 1 (rGOT) has been shown to enable the scavenging of excess excitotoxic glutamate which accumulates in the brain following a number of medical indications which are known to release excess glutamate in the brain including ischemic stroke, traumatic brain injuries and glioma brain tumors.

Excess glutamate in the brain is one of the leading causes for neurological damage. The invasive nature and rapid growth of brain tumors of the Glioblastoma Multiforme (GBM) type are considered incurable. One of the reasons for their rapid growth and for the neurological damage that is associated with the disease is that GBM cells continuously secrete excitotoxic glutamate molecules which accumulate in the brain causing the death of normal brain cells adjacent to the tumor. This, in turn, also creates the intracranial space needed for the rapid tumor expansion.

Co-administration of rGOT1 and the standard therapeutic drug Temozolomide (TMZ) in a GBM mouse model significantly retarded tumor growth, and extended their life span from 45 to 76 days compared to the group that received TMZ treatment alone (Ruban A, et al., Invest New Drugs (2012) 30(6): 2226-2235).

It has previously shown that reduction of blood glutamate levels caused by the administration of rGOT1 in an Ischemic Stroke rat model, results in the rapid efflux (within 15 min) of excess, excytotoxic glutamate molecules accumulated in the brain, through the blood brain barrier into the blood stream (F. Campos, et al., Journal of Cereb. Blood Flow Metabol. 2011, 31, 1387; M. Perez-Mato, et al., Cell Death & Dis. 2014, 5, e992).

Additional background art includes Prakash et al. Microbial Cell Factories 2012, 11:92; Marblestone et al., Protein Sci. 2006 January; 15(1): 182-189; and US Patent Application No. 20100021987.

### SUMMARY OF THE INVENTION

According to an aspect of some embodiments of the present invention there is provided a protein preparation comprising Glutamate Oxaloacetate Transaminase 1 (GOT1) polypeptide molecules, wherein 100% of the GOT1 polypeptide molecules have an alanine at position 1 of the GOT1 polypeptide, and wherein the GOT1 polypeptide molecules constitute at least 95% of the proteins in the preparation.

According to an aspect of some embodiments of the present invention there is provided a pharmaceutical composition comprising the protein preparation described herein as the active agent and a pharmaceutically acceptable carrier.

According to an aspect of some embodiments of the present invention there is provided a fusion protein comprising a polypeptide of interest and Small Ubiquitin-like Modifier (SUMO), wherein the N terminal of the polypeptide of interest is translationally fused to the C terminal of the SUMO, wherein the fusion protein is devoid of a heterologous affinity tag.

According to an aspect of some embodiments of the present invention there is provided a Glutamate Oxaloacetate Transaminase 1 (GOT1) fusion protein comprising GOT1 and Small Ubiquitin-like Modifier (SUMO), wherein the N terminal of the GOT1 is translationally fused to the C terminal of the SUMO, wherein the fusion protein is devoid of an affinity tag.

According to an aspect of some embodiments of the present invention there is provided an isolated polynucleotide encoding the fusion protein described herein.

According to an aspect of some embodiments of the present invention there is provided a nucleic acid construct comprising the isolated polynucleotide described herein.

According to an aspect of some embodiments of the present invention there is provided a method of treating a disease or condition associated with an excess of glutamate in a subject in need thereof, comprising administering to the subject a therapeutically effective amount of the preparation or pharmaceutical described herein, thereby treating the disease or condition.

According to an aspect of some embodiments of the present invention there is provided a method of purifying a polypeptide comprising:

(a) expressing a fusion protein comprising the polypeptide and SUMO in host cells, wherein the N terminal of the polypeptide is translationally fused to the C terminal of the SUMO, wherein the fusion protein is devoid of a heterologous affinity tag;

(b) removing the SUMO from the fusion protein; and

(c) isolating the polypeptide from the host cells, thereby purifying the polypeptide.

According to some embodiments of the invention, the protein constitutes at least 98% of the molecules in the preparation.

According to some embodiments of the invention, the GOT1 comprises an amino acid sequence at least 90% homologous to SEQ ID NO: 2.

According to some embodiments of the invention, the GOT1 consists of the amino acid sequence as set forth in SEQ ID NO: 2.

According to some embodiments of the invention, the GOT1 comprises an alanine at position 1.

According to some embodiments of the invention, the affinity tag is a polyhistidine tag.

According to some embodiments of the invention, the amino acid sequence of the GOT1 is at least 90% homologous to SEQ ID NO: 2.

According to some embodiments of the invention, the amino acid sequence of the SUMO is at least 90% homologous to SEQ ID NO: 5.

According to some embodiments of the invention, the fusion protein comprises an amino acid sequence at least 90% homologous to SEQ ID NO: 1.

According to some embodiments of the invention, the amino acid sequence is as set forth in SEQ ID NO: 1.

According to some embodiments of the invention, the SUMO is a yeast SUMO.

According to some embodiments of the invention, the yeast SUMO is Saccharomyces cerevisiae suppressor of mif two 3(Smt3).

According to some embodiments of the invention, the isolated polynucleotide comprises a nucleic acid sequence at least 90% homologous to SEQ ID NO: 3.

According to some embodiments of the invention, the disease or condition s a brain disease or condition.

According to some embodiments of the invention, the brain disease is a cancer of the central nervous system.

According to some embodiments of the invention, the cancer is a glioblastoma.

According to some embodiments of the invention, the brain condition is cerebral ischemia.

According to some embodiments of the invention, the disease is a neuordegenerative disease.

According to some embodiments of the invention, the step (b) is effected prior to step (c).

According to some embodiments of the invention, the polypeptide is GOT1.

According to some embodiments of the invention, the removing is effected using a SUMO protease.

According to some embodiments of the invention, the isolating is effected using a technique selected from the group consisting of heat precipitation, salt induced precipitation, mixed mode chromatography, cation exchange chromatography and anion exchange chromatography.

According to some embodiments of the invention, the isolating is effected using heat precipitation, salt induced precipitation, mixed mode chromatography, cation exchange chromatography and anion exchange chromatography.

According to some embodiments of the invention, the isolating is effected by:

(a) purifying the GOT1 by heat treatment;

(b) purifying the GOT1 by salt induced precipitation;

(c) purifying the GOT1 by mixed mode chromatography;

(d) purifying the GOT1 by cation exchange chromatography; and

(e) purifying the GOT1 by anion exchange chromatography, wherein step (e) follows step (d), wherein step (d) follows step (c), wherein step (c) follows step (b) and wherein step (b) follows step (a).

Heat precipitation, salt-induced precipitation comprises ammonium sulphate induced precipitation.

Heat precipitation, host cells are selected from the group consisting of bacteria, yeast, mammalian cells, and insect cells.

According to some embodiments of the invention, the host cells are bacterial cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 shows the full scheme of the SUMO-GOT biosynthesis process.

FIG. 2 pH, pO2, stirrer, temperature, airflow and culture optical density monitoring in fermenter during biosynthesis.

FIG. 3 is a typical SDS-PAGE representation of biomass obtained during chemically defined medium and high density.

FIG. 4 is a flow chart of the purification scheme.

FIG. 5 shows a typical chromatography profile of PPA Hyper Cel.

FIG. 6 shows a typical chromatography profile of CM Sepharose FF.

FIG. 7 shows a typical chromatography profile of Q Sepharose FF.

FIG. 8 is a photograph of a SDS-PAGE titration. Line 3—2.5 μg; line 5—5.0 μg; line 7—10.0 μg; line 9—20 μg.

FIG. 9 shows a typical RP-HPLC chromatography profile of final GOT1 solution

FIG. 10 shows a typical SEC-HPLC chromatography profile of final GOT1 solution.

FIG. 11 Overlaid chromatograms of control and GOT1 samples peptide mapping.

FIG. 12 is a photograph of a Coomassie-stained gel illustrating impurities by Isoelectric Focusing. 1—Broad range pI marker; 2—Reference solution A; 3—Reference solution B; 4—Batch 1; 5—Batch 2; 6—Batch 3; 7—Batch 4; 8—Batch 5; 9—Broad range pI marker.

### DESCRIPTION OF SPECIFIC EMBODIMENTS OF THE INVENTION

The present invention, in some embodiments thereof, relates to purified Glutamate Oxaloacetate Transaminase 1 (GOT1) preparations and to methods of generating same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Blood glutamate scavenging is an attractive protecting strategy to reduce the excitotoxic effect of extracellular glutamate released during various disorders including Glioblastoma Multiforme (GBM) and ischemic brain injury.

Until presently, recombinant human rGOT was prepared in an E. coli system which included a Histidine-6 tag that is typically used for convenient direct affinity purification of the recombinant protein. Since His-tagged containing recombinant proteins cannot be used in humans due to immunological implications, there was a need to produce an rGOT 1 preparation identical to the human enzyme, which has in its N-terminal position an Alanine moiety (J M, Doyle et al. The amino acid sequence of cytosolic aspartate aminotransferase from human liver. Biochem. J. 1990, 270: 651-7).

Recombinant bacterial expression systems generate recombinant polypeptides which comprise an N-terminal methionine. This amino acid, depending on the sequence of the next five N-terminal amino acids, may be cleaved by methionine amino peptidase (MAP2). However, it was found that during cleavage of the N-terminal methionine from rGOT1 using MAP2 bacterial enzyme, not only was the N-terminal methionine cleaved by this enzyme, but also the next three amino acids (alanine-proline-proline) which became exposed following methionine cleavage. Thus, a heterogeneous protein population was produced, with some members containing N-terminal alanine, whilst other members having been cleaved at the second or third N-terminal amino acids. Such heterogeneous protein preparations are not suitable for human therapy.

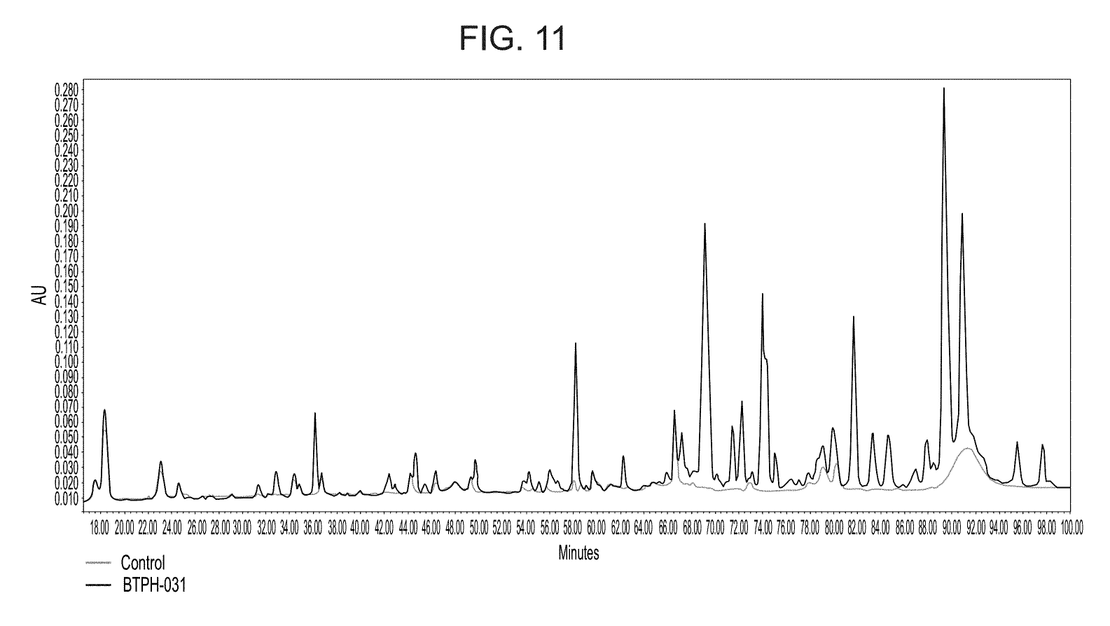

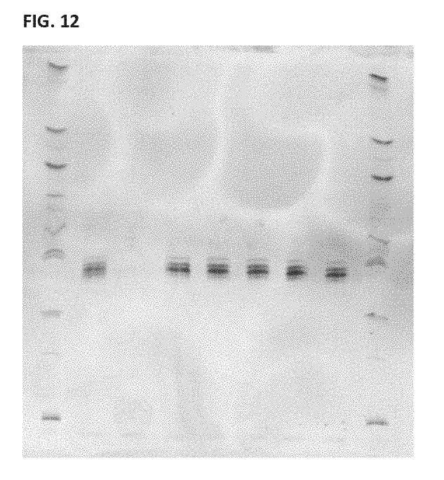

To solve this problem, the present inventors generated a chimeric protein by seamlessly fusing the GOT1 gene with the gene encoding the SUMO entity (Smt3, yeast S. cerevisiae origin). Following expression in a bacterial system, cleavage of the SUMO entity from the fusion protein, and biochemical purification of the rGOT1, the present inventors obtained a biologically active rGOT1 preparation of over 95% purity, as illustrated in FIGS. 11 and 12 and Table 6, herein below.

Thus, according to a first aspect of the present invention there is provided a method of purifying a polypeptide comprising:

(a) expressing a fusion protein comprising the polypeptide and SUMO in host cells, wherein the N terminal of the polypeptide is translationally fused to the C terminal of the SUMO, wherein the fusion protein is devoid of a heterologous affinity tag;

(b) removing the SUMO from the fusion protein; and

(c) isolating the polypeptide from the host cells, thereby purifying the polypeptide.

The term “fusion protein” refers to a protein that includes polypeptide components derived from more than one parental protein or polypeptide. The fusion protein of this aspect of the present invention is expressed from a fusion gene in which a nucleotide sequence encoding a polypeptide sequence from one protein of interest is appended in frame (and without a linker) with a nucleotide sequence encoding a polypeptide sequence of SUMO. The fusion gene can then be expressed by a recombinant host cell as a single protein, as further described herein below.

According to this aspect of the present invention, the fusion protein is devoid of heterologous affinity purification tags as further detailed herein below.

As used herein the term “SUMO” refers to a polypeptide that is a member of the Small Ubiquitin-like Modifier (or SUMO) protein family. SUMO proteins are typically small; most are around 100 amino acids in length and 12 kDa in mass. The exact length and mass varies between SUMO family members and depends on which organism the protein comes from. The SUMO may be a yeast SUMO (e.g. Saccharomyces cerevisiae suppressor of mif two 3(Smt3)), human SUMO-1, human SUMO-2, human SUMO-3, any one of Arabidopsis thalania SUMO-1 through SUMO-8, tomato SUMO, any one of Xenopus laevis SUMO-1 through SUMO-3, Drosophila melanogaster Smt3, Caenorhabditis elegans SMO-1, Schizosaccharomyces pombe Pmt3, malarial parasite Plasmodium falciparum SUMO, mold Aspergillus nidulans SUMO.

According to a particular embodiment, the SUMO protein is a Saccharomyces cerevisiae SUMO (Smt3) and is at least 90% homologous, at least 91% homologous, at least 92% homologous, at least 93% homologous, at least 94% homologous, at least 95% homologous, at least 96% homologous, at least 97% homologous, at least 98% homologous, at least 99% homologous and even more preferably 100% homologous to the amino acid sequence as set forth in SEQ ID NO: 5, as determined using the Standard protein-protein BLAST [blastp] software of the NCBI.

The second polypeptide (or the polypeptide of interest) of the fusion protein of this aspect of the present invention may be any polypeptide employed in research and industrial settings, for example, for production of therapeutics, vaccines, diagnostics, biofuels, and many other applications of interest.

The polypeptides may be intracellular polypeptides (e.g., a cytosolic protein), transmembrane polypeptides, or secreted polypeptides. The polypeptides may be full length polypeptides or fragments thereof. According to one embodiment the polypeptides are less than 10 amino acids, 20 amino acids, 50 amino acids or 100 amino acids.

According to a particular embodiment, the polypeptides are human polypeptides and have amino acid sequences which are at least 95% homologous, more preferably 96% homologous, 97% homologous 98% homologous, 99% homologous and even more preferably 100% homologous to the amino acid sequence of the wild-type protein, as determined using the Standard protein-protein BLAST [blastp] software of the NCBI.

Exemplary therapeutic proteins that can be produced by employing the subject compositions and methods include but are not limited to certain native and recombinant human hormones (e.g., insulin, growth hormone, insulin-like growth factor 1, follicle-stimulating hormone, and chorionic gonadotrophin), hematopoietic proteins (e.g., erythropoietin, C-CSF, GM-CSF, and IL-11), thrombotic and hematostatic proteins (e.g., tissue plasminogen activator and activated protein C), immunological proteins (e.g., cytokines, chemokines, lymphokines), antibodies and other enzymes (e.g., deoxyribonuclease I). Exemplary vaccines that can be produced by the subject compositions and methods include but are not limited to vaccines against various influenza viruses (e.g., types A, B and C and the various serotypes for each type such as H5N2, H1N1, H3N2 for type A influenza viruses), HIV, hepatitis viruses (e.g., hepatitis A, B, C or D), Lyme disease, and human papillomavirus (HPV). Examples of heterologously produced protein diagnostics include but are not limited to secretin, thyroid stimulating hormone (TSH), HIV antigens, and hepatitis C antigens.

Examples of specific polypeptides or proteins include, but are not limited to granulocyte macrophage colony stimulating factor (GM-CSF), granulocyte colony stimulating factor (G-CSF), macrophage colony stimulating factor (M-CSF), colony stimulating factor (CSF), interferon beta (IFN-beta), interferon gamma (IFNgamma), interferon gamma inducing factor I (IGIF), transforming growth factor beta (IGF-beta), RANTES (regulated upon activation, normal T-cell expressed and presumably secreted), macrophage inflammatory proteins (e.g., MIP-1-alpha and MIP-1-beta), Leishmnania elongation initiating factor (LEIF), platelet derived growth factor (PDGF), tumor necrosis factor (TNF), growth factors, e.g., epidermal growth factor (EGF), vascular endothelial growth factor (VEGF), fibroblast growth factor, (FGF), nerve growth factor (NGF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), TNF alpha type II receptor, erythropoietin (EPO), insulin and soluble glycoproteins e.g., gp120 and gp160 glycoproteins. Other examples include secretin, nesiritide (human B-type natriuretic peptide (hBNP)) and GYP-I.

Other exemplary polypeptides are disclosed in U.S. Application No. 2012032909, the contents of which are incorporated herein by reference.

In certain embodiments, the heterologously produced protein is an enzyme or biologically active fragments thereof. Suitable enzymes include but are not limited to: oxidoreductases, transferases, hydrolases, lyases, isomerases, and ligases. In certain embodiments, the heterologously produced protein is an enzyme of Enzyme Commission (EC) class 1, for example an enzyme from any of EC 1.1 through 1.21, or 1.97. The enzyme can also be an enzyme from EC class 2, 3, 4, 5, or 6. For example, the enzyme can be selected from any of EC 2.1 through 2.9, EC 3.1 to 3.13, EC 4.1 to 4.6, EC 4.99, EC 5.1 to 5.11, EC 5.99, or EC 6.1-6.6.

According to a particular embodiment the polypeptide is Glutamic-oxaloacetic transaminase, type 1 (GOT1; NM_002079.2; P17174). This enzyme is a pyridoxal phosphate (PLP)-dependent cytoplasmic enzyme having an EC No. 2.6.1.1. GOT1 plays a role in amino acid metabolism and the urea and tricarboxylic acid cycles. The aspartate aminotransferase activity is involved in hepatic glucose synthesis during development and in adipocyte glyceroneogenesis. GOT1 is also an important regulator of levels of serum glutamate. The normal brain has very low levels of extracellular glutamate (about 1 micromolar) in contrast with the high level of glutamate present in the blood circulation (about 40 micromolar). The small amount of brain glutamate plays an important role as a neurotransmitter of the vertebrate central nervous system.

The GOT1 of the fusion protein of this aspect of the present invention has an amino acid sequence of serum GOT1 (i.e. comprises an alanine at its N terminus (at position 1 of the protein sequence).

Preferably, the GOT1 comprises an amino acid sequence at least 90% homologous, at least 91% homologous, at least 92% homologous, at least 93% homologous, at least 94% homologous, at least 95% homologous, at least 96% homologous, at least 97% homologous, at least 98% homologous, at least 99% homologous and even more preferably 100% homologous to the amino acid sequence as set forth in SEQ ID NO: 2, as determined using the Standard protein-protein BLAST [blastp] software of the NCBI (wherein the first amino acid of the protein is alanine and not methionine).

According to a specific embodiment, the GOT1 consists of a sequence as set forth in SEQ ID NO: 2.

Preferably, the SUMO-GOT1 fusion protein comprises an amino acid sequence at least 90% homologous, at least 91% homologous, at least 92% homologous, at least 93% homologous, at least 94% homologous, at least 95% homologous, at least 96% homologous, at least 97% homologous, at least 98% homologous, at least 99% homologous and even more preferably 100% homologous to the amino acid sequence as set forth in SEQ ID NO: 1, as determined using the Standard protein-protein BLAST [blastp] software of the NCBI.

According to another embodiment, the SUMO-GOT1 fusion protein consists of the amino acid sequence as set forth in SEQ ID NO: 1.

To produce a SUMO fusion protein (e.g. SUMO-GOT1 fusion protein) using recombinant technology, an isolated polynucleotide comprising a nucleic acid sequence encoding such a polypeptide may be used. An exemplary nucleic acid sequence is set forth in SEQ ID NO: 3.

The term “nucleic acid sequence” refers to a deoxyribonucleic acid sequence composed of naturally-occurring bases, sugars and covalent internucleoside linkages (e.g., backbone) as well as oligonucleotides having non-naturally-occurring portions which function similarly to respective naturally-occurring portions. Such modifications are enabled by the present invention provided that recombinant expression is still allowed.

A nucleic acid sequence of the fusion protein according to this aspect of the present invention can be a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Linking of a polynucleotide sequence which encodes SUMO and a polynucleotide sequence that encodes a protein of interest may be effected using standard molecular biology techniques including the use of PCR, ligation enzymes and restriction enzymes. It will be appreciated that the 5′ end of the SUMO is ligated to the 3′ end of the gene encoding the protein of interest such that a fusion protein is generated with SUMO at the N terminus and the polypeptide of interest at the C terminus.

The generated polynucleotide which encodes the fusion protein is typically devoid of any sequence encoding a heterologous affinity tag.

The phrase “heterologous affinity tag” as used herein, refers to an amino acid sequence that is not naturally comprised in SUMO or the polypeptide of interest (i.e. in the wild-type sequences) that can be used to affinity purify the fusion protein or polypeptide of interest.

Thus, for example, the isolated polynucleotides of the this aspect of the present invention are devoid of nucleic acid sequences encoding polyhistidine tags, polyarginine tags, glutathione-S-transferase, maltose binding protein, S-tag, influenza virus HA tag, thioredoxin, staphylococcal protein A tag, the FLAG™ epitope, AviTag epitope, and the c-myc epitope.

In order to generate the fusion proteins of the present invention using recombinant techniques, the polynucleotides encoding same are ligated into nucleic acid expression vectors, such that the polynucleotide sequence is under the transcriptional control of a cis-regulatory sequence (e.g., promoter sequence).

A variety of prokaryotic or eukaryotic cells can be used as host-expression systems to express the polypeptides of the present invention. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

Exemplary bacterial cells that may be used to express the fusion protein are E. coli, such as an E. coli protein deficient strain, e.g. E. coli BL21 or Rosetta gami-2, most preferably an E. coli BL21.

Exemplary yeast cells that may be used to express the fusion protein are K. lactis or S. cerevisiae.

Constitutive promoters suitable for use with this embodiment of the present invention include sequences which are functional (i.e., capable of directing transcription) under most environmental conditions and most types of cells such as the cytomegalovirus (CMV) and Rous sarcoma virus (RSV).

Inducible promoters suitable for use with this embodiment of the present invention include for example the tetracycline-inducible promoter (Srour, M. A., et al., 2003. Thromb. Haemost. 90: 398-405) or the lac operator. In the latter case, gene expression is induced using Isopropyl β-D-1-thiogalactopyranoside (IPTG) at a concentration between 0.1 mM-1 mM at a temperature between 20-30° C. (for example 25° C.).

The expression vector according to this embodiment of the present invention may include additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

Polyadenylation sequences can also be added to the expression vector in order to increase the translation efficiency of a polypeptide expressed from the expression vector of the present invention. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples of bacterial expression vectors suitable for the present invention include but are not limited to pET21b+, pBR322 or pET28+.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can also be used by the present invention. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMT010/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins.

Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the recombinant cell, secreted into the fermentation medium, secreted into a space between two cellular membranes, such as the periplasmic space in E. coli; or retained on the outer surface of a cell or viral membrane.

Following a predetermined time in culture, recovery of the recombinant fusion protein is effected.

If the fusion protein is expressed in the cell, the cell membrane is preferably disrupted so as to release the fusion protein.

Cell disruption may be effected using methods known in the art including homogenization.

The fusion protein is desumoylated by the addition of SUMO protease (EC 3.4.22.68).

Desumoylation may be effected at any stage in the purification procedure. According to a particular embodiment, this procedure is effected following heat denaturation and prior to salt precipitation and/or exchange chromatography, as further described herein below.

The SUMO protease may comprise Saccharomyces cerevisiae ULP1 (Ubl-specific protease 1) from Saccharomyces cerevisiae, Ulp2, human SENP1 human SENP2, human SENP3, human SEN P5, human SENP6 human SEN P7, mouse SENP1, mouse SENP2, mouse SENP3, mouse SEN P5, mouse SENP6, mouse SENP7, any one of Arabidopsis thalania Ulp1a through Ulp1d, any one of Arabidopsis thalania Ulp2a through Ulp2h, Arabidopsis ihalania ESD4, Caenorhabditis elegans Ulp-1, Caenorhanditis elegans Ulp-2, Drosophila melanogaster ULP1, Schizosaccharomyces pombe Ulp1, Schizosaccharomyces pombe ULP2, Aspergillus nidulans Ulp, Xenopus laevis XSENP1a, Xenopus laevis XSENP1b, Xanthomonas campestris XopD, Kluyveromyces lactis Ulp1, Plasmodium falciparum Ulp, an equivalent thereof, a homologue thereof, a catalytic domain thereof, or a combination thereof.

The fusion protein can be purified using any method known in the art including, but not limited to heat denaturation, salt induced precipitation, mixed mode chromatography, cation exchange chromatography and anion exchange chromatography.

Heat Denaturation:

Different proteins have different stabilities at elevated temperatures, and if the target protein has a greater heat stability than contaminating proteins, incubation at elevated temperatures for periods of time, varying from a few minutes to a few hours, often precipitates unwanted proteins, which can then be removed by centrifugation. The stability of the target protein at elevated temperatures may in some cases be enhanced by the presence of substrates or other specific ligands that enhance folding thereof. GOT1 in the presence of alfa-ketoglutarate (e.g. 10 mM) and pyridoxal 5-phosphate (e.g. 20 μM) is heat stable at 70° C. Accordingly by increasing the temperature to about 70° C., contaminating proteins that are denatured may be precipitated.

Salt Induced Precipitation:

The solubility of proteins varies according to the ionic strength of the solution, and hence according to the salt concentration. Two distinct effects are observed: at low salt concentrations, the solubility of the protein increases with increasing salt concentration (i.e. increasing ionic strength), an effect termed salting in. As the salt concentration (ionic strength) is increased further, the solubility of the protein begins to decrease. At sufficiently high ionic strength, the protein will be almost completely precipitated from the solution (salting out). Since proteins differ markedly in their solubilities at high ionic strength, salting-out (or salt—induced precipitation is a very useful procedure to assist in the purification of a given protein. The commonly used salt is ammonium sulfate, as it is very water soluble, forms two ions high in the Hofmeister series, and has no adverse effects upon enzyme activity. It is generally used as a saturated aqueous solution which is diluted to the required concentration, expressed as a percentage concentration of the saturated solution (a 100% solution).

Chromatography Resin:

The term “chromatography resin” or “chromatography media” are used interchangeably herein and refer to any kind of solid phase which separates an analyte of interest (e.g., the polypeptide of interest or the fusion protein of the present invention) from other molecules present in a mixture. Usually, the analyte of interest is separated from other molecules as a result of differences in rates at which the individual molecules of the mixture migrate through a stationary solid phase under the influence of a moving phase, or in bind and elute processes. Non-limiting examples include cation exchange resins, anion exchange resins and mixed mode resins. The volume of the resin, the length and diameter of the column to be used, as well as the dynamic capacity and flow-rate depend on several parameters such as the volume of fluid to be treated, concentration of protein in the fluid to be subjected to the process of the invention, etc. Determination of these parameters for each step is well within the average skills of the person skilled in the art.

Mixed Mode Chromatography:

Mixed mode chromatography is chromatography that utilizes a mixed mode of chromatography ligands. In certain embodiments, such a ligand refers to a ligand that is capable of providing at least two different, but co-operative, sites which interact with the substance to be bound. One of these sites gives an attractive type of charge-charge interaction between the ligand and the substance of interest. The other site typically gives electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole etc.

In certain embodiments, the mixed mode chromatography media is comprised of mixed mode ligands coupled to an organic or inorganic support, sometimes denoted a base matrix, directly or via a spacer. The support may be in the form of particles, such as essentially spherical particles, a monolith, filter, membrane, surface, capillaries, etc. In certain embodiments, the support is prepared from a native polymer, such as cross-linked carbohydrate material, such as agarose, agar, cellulose, dextran, chitosan, konjac, carrageenan, gellan, alginate etc. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964). Alternatively, the support can be prepared from a synthetic polymer, such as cross-linked synthetic polymers, e.g. styrene or styrene derivatives, divinylbenzene, acryl amides, acrylate esters, methacrylate esters, vinyl esters, vinyl amides etc. Such synthetic polymers can be produced according to standard methods, see e.g. “Styrene based polymer supports developed by suspension polymerization” (R Arshady: Chimica e L'Industria 70(9), 70-75 (1988)). Porous native or synthetic polymer supports are also available from commercial sources, such as GE healthcare, Uppsala, Sweden.

In certain embodiments, the mixed-mode resin comprises functional groups capable of anionic exchange and hydrophobic interactions. Examples of such resins include, but are not limited to Butyl-Sepharose, Octyl-Sepharose, Phenyl-Sepharose, (e.g. in pH range of 7-9), MEP HyperCel, PPA HyperCel (e.g. in pH range 4.0-8.0), HEA HyperCel (all manufactured by Pall Corp.). Capto Adhere or Capto MMC.

Cation Exchange Chromatography:

In performing the separation, the protein mixture can be contacted with the cation exchange material by using any of a variety of techniques, e.g., using a batch purification technique or a chromatographic technique. Proteins which have an overall positive charge when present in a buffer having a pH below the protein's pI, will bind well to cation exchange material, which contain negatively charged functional groups. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution). Cationic substituents may be attached to matrices in order to form cationic supports for chromatography. Non-limiting examples of cationic exchange substitutents include carboxymethyl (CM), sulfoethyl(SE), sulfopropyl(SP), phosphate(P) and sulfonate(S).

Preferably, the CEX resin comprises a CM functional group.

Cellulose ion exchange resins such as DE23™, DE32™, DE52™, CM-23™, CM-32™, and CM-52™ are available from Whatman Ltd. Maidstone, Kent, U.K. SEPHADEX®-based and cross-linked ion exchangers are also known. For example, DEAE-, QAE-, CM-, and SP-SEPHADEX® and DEAE-, Q-, CM- and S-SEPHAROSE® and SEPHAROSE® Fast Flow are all available from Pharmacia AB. Further, both DEAE and CM derivitized ethylene glycol-methacrylate copolymer such as TOYOPEARL™ DEAE-650S or M and TOYOPEARL™ CM-650S or M are available from Tosoh, Philadelphia, Pa.

Further, both DEAE and CM derivitized ethylene glycol-methacrylate copolymer such as TOYOPEARL™ DEAE-650S or M and TOYOPEARL™ CM-650S or M are available from Tosoh, Philadelphia, Pa., or Nuvia S and U OSphere™ S from BioRad, Hercules, Calif., Eshmuno® S from EMD Millipore, Billerica, Calif.

Anion Exchange Chromatography:

Various anionic exchange chromatography supports can be used and may be selected from the group consisting of: DEAE-Sepharose CL-6B, DEAE-Sepharose FF, Q-Sepharose FF, Q-Sepharose HP, Q-Sepharose XL, DEAE-Sephacel, DEAE-Sephadex, QAE-Sephadex, DEAE-Toyopearl, QAE-Toyopearl, Mini-Q, Mono-Q, Mono-P, Source 15Q, Source 30Q, ANX-Sepharose etc. Preferably, the anionic exchange chromatography is performed on Q-sepharose or DEAE-Sepharose (e.g. in pH range of 4.0-9.0).

According to a particular embodiment, when the protein is GOT1, the purifying is effected by the following techniques in the order as disclosed:

(a) purifying the GOT1 by heat treatment (e.g. 65-70° C. for 10 min);

(b) purifying the GOT1 by salt induced precipitation (e.g. ammonium sulphate precipitation);

(c) purifying the GOT1 by mixed mode chromatography (e.g. PPA HyperCel at a pH range of 4.0-8.0);

(d) purifying the GOT1 by cation exchange chromatography (e.g. SP-Sepharose or CM-Sepharose at a pH range of 4.0-7.0; and

(e) purifying the GOT1 by anion exchange chromatography (e.g. Q-Sepharose or DEAE-Sepharose in pH range of 4.0-9.0).

Using the methods described herein, the present inventors generated protein preparations of highly purified GOT1 having alanine (and not methionine) at position 1 of the protein.

Thus, according to another aspect of the present invention there is provided a protein preparation comprising Glutamate Oxaloacetate Transaminase 1 (GOT1) polypeptide molecules, wherein 100% of the GOT1 polypeptide molecules have an alanine at position 1 of the GOT1 polypeptide, and wherein the GOT1 polypeptide molecules constitute at least 95% of the proteins in the preparation.

As used herein, the phrase “protein preparation” refers to a liquid mixture of at least one protein, e.g. a cell lysate, a partial cell lysate which contains not all proteins present in the original cell or a combination of several cell lysates. The term “protein preparation” also includes dissolved purified protein. Typically, a protein preparation is devoid of other cell components such as lipids, fats, nucleic acids etc.

The GOT1 polypeptide is present in a solubilized state in the protein preparation.

According to one embodiment, at least 90% of the proteins in the preparation are GOT1 polypeptide molecules.

According to one embodiment, at least 91% of the proteins in the preparation are GOT1 polypeptide molecules.

According to one embodiment, at least 92% of the proteins in the preparation are GOT1 polypeptide molecules.

According to one embodiment, at least 93% of the proteins in the preparation are GOT1 polypeptide molecules.

According to one embodiment, at least 94% of the proteins in the preparation are GOT1 polypeptide molecules.

According to one embodiment, at least 95% of the proteins in the preparation are GOT1 polypeptide molecules.

According to one embodiment, at least 96% of the proteins in the preparation are GOT1 polypeptide molecules.

According to one embodiment, at least 97% of the proteins in the preparation are GOT1 polypeptide molecules.

According to one embodiment, at least 98% of the proteins in the preparation are GOT1 polypeptide molecules.

According to one embodiment, at least 99% of the proteins in the preparation are GOT1 polypeptide molecules.

According to one embodiment, 100% of the proteins in the preparation are GOT1 polypeptide molecules.

The GOT1 polypeptides in the protein preparations have an alanine at the N terminal (at position 1 of the protein).

According to this aspect of the present invention the percent of GOT1 proteins in the preparation which have an alanine at position 1 of the protein is higher when compared to preparations of recombinant GOT1 proteins which were expressed in bacterial cells, wherein the methionine is cleaved using a methionine amino peptidase.

Thus, the percent of GOT1 proteins in the preparation of this aspect of the present invention may be 1% higher, 2% higher, 3% higher, 4% higher, 5% higher or more when compared to preparations of recombinant GOT1 proteins which were expressed in bacterial cells, wherein the methionine is cleaved using a methionine amino peptidase.

According to this aspect of the present invention at least 99.9% of the GOT1 molecules comprise alanine at position 1, at least 99.99% of the GOT1 molecules comprise alanine at position 1, at least 99.999% of the GOT1 molecules comprise alanine at position 1 and preferably at least 99.9999% or 100% of the GOT1 molecules comprise alanine at position 1.

The recombinant GOT I polypeptides described herein may be used to treat diseases or conditions associated with an excess of glutamate.

Examples of diseases associated with excess glutamate include but are not limited to brain anoxia, stroke, perinatal brain damage, traumatic brain injury, bacterial meningitis, subarachnoid hemorrhage, epilepsy, acute liver failure, glaucoma, amyotrophic lateral sclerosis, HIV, dementia, amyotrophic lateral sclerosis (ALS), spastic conditions, open heart surgery, aneurism surgery, coronary artery bypass grafting and Alzheimer's disease.

According to a particular embodiment, the disease is a cancer of the central nervous system.

As used herein the phrase “cancer of the central nervous system” refers to a brain tumor (primary or secondary), which typically releases glutamate at levels sufficient to allow glutamate to exert excitotoxicity on neighboring healthy neuronal cells. Thus, a cancer of the central nervous system include, primary tumors of glial, neuronal, schwann cell, pinealcyte, menningioma and melanoma, as well as sarcoma, lymphoma and multiple systemic malignancies that metastasize in the brain.

Specific examples include, but are not limited to, astrocytoma and glioblastoma, and also their related neural and glial tumors, glioma (e.g., which include grades 1 and 2), oligodendroglioma, neurocytoma, dysplastic neuroepithelial tumor, primitive neuroectodermal tumor, and ganglioneuroma.

Additional conditions that may be treated with the GOT-1 include traumatic brain injuries and concussions, recurrent migraine headaches, Cerebral palsy, resuscitation and asphyxia, recurrent epileptic attacks, Amyotropic lateral sclerosis (ALS), bacterial meningitis and Parkinson's disease.

The GOT1 polypeptide may be provided per se or as part of a pharmaceutical composition. As used herein a “pharmaceutical composition” refers to a preparation of one or more of the active ingredients described hereinabove along with other components such as physiologically suitable carriers and excipients, penetrants etc. The purpose of a pharmaceutical composition is to facilitate administration of a compound to an organism.

Herein the term “active ingredient” refers to the preparation accountable for the biological effect (e.g., GOT1). Hereinafter, the phrases “physiologically acceptable carrier” and “pharmaceutically acceptable carrier” are interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979)).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration of the pharmaceutical composition of the present invention may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, intraosseus and intraocular injections.

According to a particular embodiment, the route is a systemic mode and dosing is such that it reduces blood (plasma) glutamate levels and enhances brain-to-blood glutamate levels.

The administration mode may also depend on the status of the patient. For example, in case of a seizure, intraosseus administration or intravenal administration may be preferred.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any pharmaceutical composition used by the treatment method of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Animal models for cancer of the nervous system are readily available.

Thus, xenograft models are well suited for evaluating dose response characteristics of preclinical therapies, and for assessing the influence of tumor site on therapeutic response. Weissenberger et al J. Neurosurg. 2007 April; 106(4):652-9 have produced germline insertion of a transgene expressing v-src from the GFAP promoter (limiting the expression to astrocytes) resulting in the formation of astrocytomas. V-src activates several signal transduction pathways that are also activated in human gliomas. These GFAP/v-src gliomas are primarily either low grade or anaplastic but in some cases acquire the histologic characteristics of glioblastomas.

Guha et al. Am J Pathol. 2005 September; 167(3):859-67 have developed transgenic mice which over-expressed oncogenic H-Ras from the GFAP promoter as. Different founder lines of these mice express various levels of oncogenic Ras. The highest producers of H-Ras develop astrocytomas with all the characteristics of glioblastomas. The moderate H-Ras expressors go on to germline transmission and develop low grade and anaplastic astrocytomas with high prevalence by 3 months of age. Reilly et al. Nat. Genet. 2000 September; 26(1):109-13 have generated gliomas with astrocytic character with a combined deletion of Nf-1 and p53. Nf-1 is a RasGAP protein that down regulates Ras activity therefore, loss of Nf-1 results in elevated Ras activity. Alone, mutation of Nf-1 in all cells within the mouse results in astrogliosis, but not glioma formation; however, when combined with mutations of p53, mice develop astrocytic tumors with characteristics of glioblastomas in humans. Retroviral vector gene transfer of PDGF-B to somatic cells has been used to generate astrocytic gliomas by Uhrbom et al. Nat. Med. 2004 November; 10(11):1257-60 In these experiments, replication-competent MMLV vector systems result in the formation of various CNS tumor morphologies. The most frequent histology seen in these experiments are high grade gliomas with characteristics of glioblastomas. Somatic-cell gene transfer with tissue-specific ALV based RCAS retroviral vectors also show the formation of glioblastomas in mice. These tumors arise after combined gene transfer of genes encoding activated Ras and Akt to nestin expressing CNS progenitors. In this system, neither Ras nor Akt alone are sufficient for the generation of these glioblastomas. A transgenic mouse model with features of human WHO grade III astrocytoma was developed by astrocyte-specific inactivation of pRb and related proteins, p107 and p130 (Xiao et al., 2002). This was accomplished by expression of a single copy of the T121 gene driven by the GFAP promoter. T121 is a 121-amino acid N-terminal fragment of SV40 T antigen that dominantly inactivates the pRb proteins, but does not interfere with p53 function. One hundred percent of TgG(Z) T121 mice develop high grade astrocytoma at around 6 months of age. Histologic features resembling the human disease include adhesion to neurons and vasculature.

Depending on the severity and responsiveness of the condition to be treated, dosing can be of a single or a plurality of administrations, with course of treatment lasting from several days to several weeks or until cure is effected or diminution of the disease state or symptoms is achieved.

The amount of the pharmaceutical composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack or an automatic syringe (PEN) with a prefilled dose for personal daily injection by the patient). The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that the present agents can be provided (as adjuvant therapy) along with other treatment modalities for brain tumors, which are selected based on location, the cell type and the grade of malignancy. Conventional therapies include surgery, radiation therapy, and chemotherapy. Temozolomide is a chemotherapeutic drug that is able to cross the blood-brain barrier effectively and is being used in therapy. For recurrent high-grade glioblastoma, recent studies have taken advantage of angiogenic blockers such as bevacizumab in combination with conventional chemotherapy, with encouraging results. Other agents include, but are not limited to, temodal, nitrosoureas, carmustine and cis-platin as well as antibody-based drugs, e.g., cetuximab.

Other anti-cancer drugs that can be co-administered with the agents of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Subjects which may be treated according to aspects of the present invention include mammalian subjects—e.g. human subjects.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521;“Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Purity by SDS-PAGE under reducing and non-reducing conditions: Purity determination of rGOT1 was performed by vertical electrophoresis in pre-cast polyacrylamide gel in the presence of the detergent sodium dodecyl sulfate (SDS) under reducing and non-reducing conditions. Samples of reduced and non-reduced protein were separated according to molecular size on polyacrylamide gel plates.

For the electrophoretical separation of rGOT1 DS samples, a NuPAGE™ 4-12% Bis-Tris Gel with MES buffer as running buffer for reducing SDS-Page and MOPS buffer for non-reducing SDS-PAGE were used. The samples were diluted with LDS NuPAGE™ Sample Buffer (4×) from Invitrogen. The samples for reducing SDS-PAGE were reduced by 0.5 M DTT. Original and prepared loading samples were gently shaken and subsequently heated. Reducing and non-reducing SDS-PAGE analyses were performed separately from each other on distinct gels.

After electrophoresis the gels were stained with PageBlue™ staining reagent and the mobility of the bands was determined.

For determination of protein purity, the intensity of each band was evaluated in respect of all bands by TotalLab software for quantitative image analysis.

Aggregates by SE-HPLC: rGOT1 dimer form was separated from its related impurities of higher molecular mass and monomer by size exclusion high performance liquid chromatography (SE-HPLC) on the basis of the different shape of the components' molecules.

Samples of the test solution were chromatographed on a stainless steel column of 300 mm length and 7.8 mm inner diameter, filled with 5 μm size hydrophylised silica gel particles suitable to fractionate globulins in 10,000-500,000 Da molecular mass intervals by using an isocratic flow of 0.05M phosphate buffer containing 0.3M NaCl, mobile phase and detection at 214 nm.

For determination of rGOT1 aggregated forms in the test solution, the percent of sum area of all peaks with retention time shorter than the dimer peak was evaluated in respect of all integrated peaks area.

Purity and related proteins by RP-HPLC: Purity of rGOT1 in respect of rGOT1 related proteins differing in their hydrophobic properties, i.e. rGOT1 oxidized/reduced and deamidated forms and other unknown related species were determined by reverse phase high performance liquid chromatography (RP-HPLC). Samples of test solution and reference solution were chromatographed on a column (250 mm×4.6 mm) packed with butyl silica gel (particle size 5 μm) with a pore size of 30 nm using a gradient of 0.1% trifluoroacetic acid in acetonitrile and detection at 215 nm. For determination of rGOT1 purity and the amount of its related substances in the test solution, the percent of each component peak area was evaluated in respect of all integrated peaks area.

Impurities by Isoelectric Focusing: Isoelectric focusing (IEF) is a method of electrophoresis that separates proteins according to their isoelectric point. Separation was carried out in a slab of polyacrylamide or agarose gel that contains a mixture of amphoteric electrolytes (ampholytes). When subjected to an electric field, the ampholytes migrate in the gel to create a pH gradient. In some cases gels containing an immobilized pH 2radient, prepared by incorporating weak acids and bases to specific regions of the gel network during the preparation of the gel, were used. When the applied proteins reach the gel fraction that has a pH that is the same as their isoelectric point (pI), their charge is neutralized and migration ceases.

Samples were run on a Precast gels from GE Healthcare under constant power for at least 1.25 hours until the standard pI markers were sharply focused. The plate was stained with Coomassie brilliant blue dye.

Protein content by UV: The protein concentration was determined by means of spectrophotometry, measuring the optical density of formulated material at 280 nm. Specific extinction coefficient (1.461ml mg⁻¹ cm⁻¹) were used for calculations of protein concentration.

The Labelguard™ Microliter cell was used for measurements. This is innovative optical pathway for protein concentration determination. The cell is designed for optimum measurement results with submicroliter sample volumes ranging from 0.7 μl up to 10 μl of undiluted sample.

Enzymatic activity by Karmen method: Enzymatic activity of rGOT1 is based on the principle of the Karmen method (Karmen A, Wroblewski F, La Due J S. Transaminase activity in human blood. J Clin Invest. 1955;34:126-31.), which incorporates a coupled enzymatic reaction using malate dehydrogenase (MD) as the indicator reaction and monitors the change in absorbance at 340 nm continuously as NAM is oxidized to NAD⁺ at 25° C. pH of reaction is 8.3.

A Unit of enzymatic activity is defined as the amount of enzyme which produced 1 μmol of oxaloacetic acid per minute at 25° C.

Peptide mapping: rGOT1 sample was prepared for digestion with endoproteinase Glu-C. The protein was denatured in order to unfold it, the disulphide bonds were cleaved and then alkylated to avoid protein refolding. After digestion, chromatographic separation was performed on a column packed with octadecyl silica gel (particle size 5 μm) with a pore size of 100 Å using a gradient elution with mobile phases consisting of 0.1% trifluoroacetic acid in water and 0.1% trifluoroacetic acid in 90% acetonitrile/water. UV detection was set up for two simultaneous wavelengths: 215 nm and 280 nm.

Visual evaluation of retention times of peptide peaks, number of peaks as well as overall elution pattern was performed. Secondly, numerical comparison of peptides in sample and Reference Standard were evaluated as percentage of component peak area in respect of all integrated peaks area as well as percentage of each peak height relative to the sum of all integrated peaks height.

PLP by Phenylhydrazine method: Pyridoxal 5′-phosphate (PLP) is an active form of vitamin B6 and is present in GOT1 as a coenzyme. PLP determination method is based on the formation of an intensely yellow hydrazine when either pyridoxal or pyridoxal 5′-phosphate are treated with phenylhydrazine (Wada H and Snell E. Journal of Biological Chemistry, 1961; 236(7), 2089-95). The formed hydrazine absorbs UV light at 410 nm. In order to determine the amount of PLP in rGOT1 sample, calibration curve in the range of 0-25 μM of PLP must be obtained. rGOT1 was diluted accordingly to the linear range of absorbance of the calibration curve, and calculated.

Molecular mass by ESI-MS: rGOT1 protein sample was prepared by desalting it with 10 mM acetic acid solution using PD MiniTrap G25 columns. For MS analysis the desalted sample was diluted 5 times by formulating it into 40% acetonitrile, 0.25% formic acid solution.

MicroTOF, Bruker mass spectrometer with Apollo Source (ESI) ionization source was used for the analysis. Molecular masses of the substances were calculated from the deconvoluted mass spectra. Homogeneity of protein N-terminus was evaluated as percentage of the particular component peak intensity in respect of all peaks intensities.

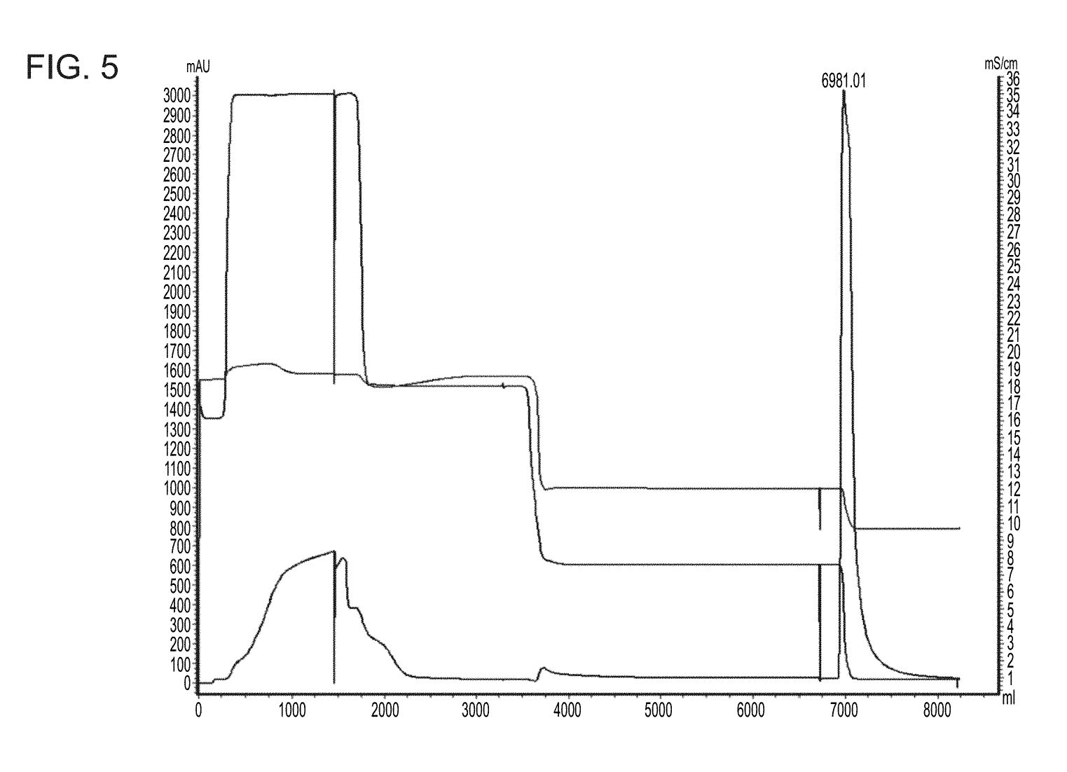

Protein N-terminus amino acid determination by Edman degradation: Cyclic degradation of peptides based on the reaction of phenylisothiocyanate with the free amino group of the N-terminal residue such that amino acids are removed one at a time and identified as their phenylthiohydantoin derivatives. The phenylthiohydantoin (PTH)-amino acid is transferred to a reversed-phase C-18 column for detection at 270 nm. A standard mixture of 19 PTH-amino acids (FIG. 5) was also injected onto the column for separation (usually as the first cycle of the sequencing run). This chromatogram provides standard retention times of the amino acids for comparison with each Edman degradation cycle chromatogram. The HPLC chromatograms were collected using a computer data analysis system. To determine the amino acid present at a particular residue number, the chromatogram from the residues of interest was compared with the chromatogram from the previous residue by overlaying one on top of the other. From this, the amino acid for the particular residue was determined.

Biosynthesis: biosynthesis was performed in Applikon bioreactors with supplied air, and pure oxygen mixture to maintain needed levels of dissolved oxygen saturation. The biosynthesis was performed in controlled temperature and pH, with continuous adaptive feeding with microelement/carbon source solution. Cultivation was performed in chemically defined medium, providing enough mixing to ensure high density process.

### Example 1

### Expression of SUMO-GOT1

A construct from E. coli BL21 (DE3) pET28/GOT1(Δhis) was used as the main matrix for GOT1 gene amplification. Primers were designed in such a manner that the full gene (SEQ ID NO: 4) was amplified omitting the initial ATG codon, coding for start-methionine. Primer sequences were as follows: Forward SUMO2: 5′-GCACCTCCGTCAGTCTTTG-3′ SEQ ID NO: 6 Reverse SUMO2: 3′-CTACTGGATTTTGGTGACTGCTTCATGGAT-5′ SEQ ID NO: 7.

The gene was amplified using Taq DNA polymerase under the following conditions: 95° C.—5 min; then repeating 30 cycles of 95° C.—1 min, 56° C.—30 s, 72° C.—1 min. Final hold at 72° C.—10 min. The amplified sequence was of 1293 bp in length (coding for the GOT1gene including restriction endonuclease targets for the fusion to the SUMO entity) and was visualized on an agarose electrophoresis gel in substantial amounts. The PCR product was then ligated using T4 DNA ligase into pET-SUMO plasmid, positive clones for orientation and size were selected for the use as matrix of the second round of PCR. Second round PCR was performed using primer sequences: SUMO-FOR-NCOI: AAA CCA TGG GAC GGA CTT AGA AGT CAA TCA A—SEQ ID NO: 8 and REV-GOT1: AAA AAG CTT CTA CTG GAT TTT GGT GAC TGC TTC A—SEQ ID NO: 9 (5′→3′orientation). Second round of PCR was performed using Pfu DNA polymerase under the conditions: 95° C.—5 min; then repeating 30 cycles of 95° C.—1 min, 58° C.—80 s, 72° C.—1 min. Final hold at 72° C.—10 min. The obtained PCR product was 1549 bp in length and contained NcoI and HindIII restriction sites for cloning into the pET28 plasmid. The second round of PCR added the SUMO entity to the GOT1 sequence as well as specific restriction sites.

Such a PCR product does not contain any affinity tags that are present in pET-SUMO plasmid (His, MYKD, etc.) and codes strictly for the SUMO entity, seamlessly fused to the GOT1 recombinant sequence. The product was cloned into the appropriately digested pET28b+ plasmid (Novagen), transformed into transient E. coli strain JM109 for initial clone selection. A clone that had correct structure confirmed both by sequencing and restriction endonuclease digestion was transformed into expression strain E. coli BL21(DE3) (purchased from Novagen). A research cell bank containing 10% of final volume glycerol was prepared from confirmed clone and used for further process development.

The E. coli BL21 (DE3) pET28/SUMO-GOT1(Δhis) strain was cultivated in a media having the following composition (g/L):

a) for inoculum preparation (cultivation) in flasks (g/L): di-sodium hydrogen phosphate (17.0), potassium dihydrogen phosphate (L82), ammonium sulfate (3.0), magnesium sulfate heptahydrate (0.5), D(+)-glucose monohydrate (15.0) and microelements stock solution (0.16 mL);

b) for fermentation (g/L): ammonium phosphate dibasic (4.0), magnesium sulfate heptahydrate (0.5), potassium dihydrogen phosphate (13.3), citric acid monohydrate (1.6), D(+)-glucose monohydrate (30.0) and microelements stock solution e) (0.25 mL);

c) feeding solution A (g/L): D(+)-glucose monohydrate (700.0), magnesium sulfate heptahydrate (20.7) and microelements stock solution e) (2.2 mL/L);

d) feeding solution B (g/L): ammonium phosphate dibasic (360.0) and potassium dihydrogen phosphate (306.7); and

e) microelements (trace elements) stock solution (g/L): iron (III) chloride hexahydrate (30.0), calcium chloride dihydrate (4.05), zinc (II) sulfate heptahydrate (6.75), manganese (II) sulfate monohydrate (1.5), copper (II) sulfate pentahydrate (3.0), cobalt (II) chloride hexahydrate (1.14), sodium molybdate dihydrate (0.3) and boric acid (0.69).

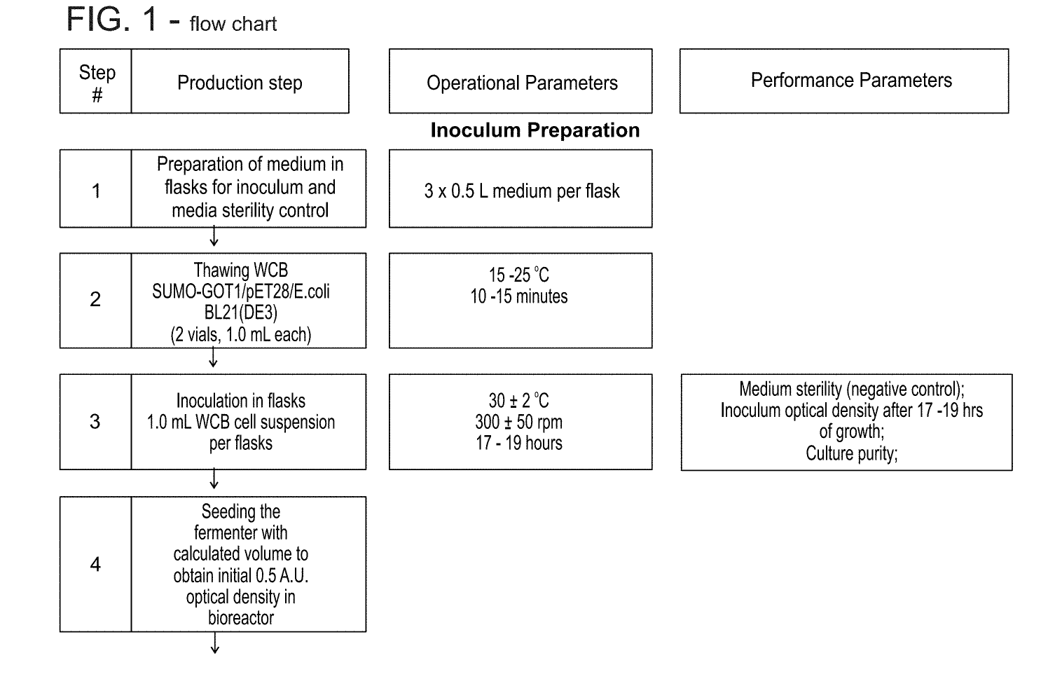

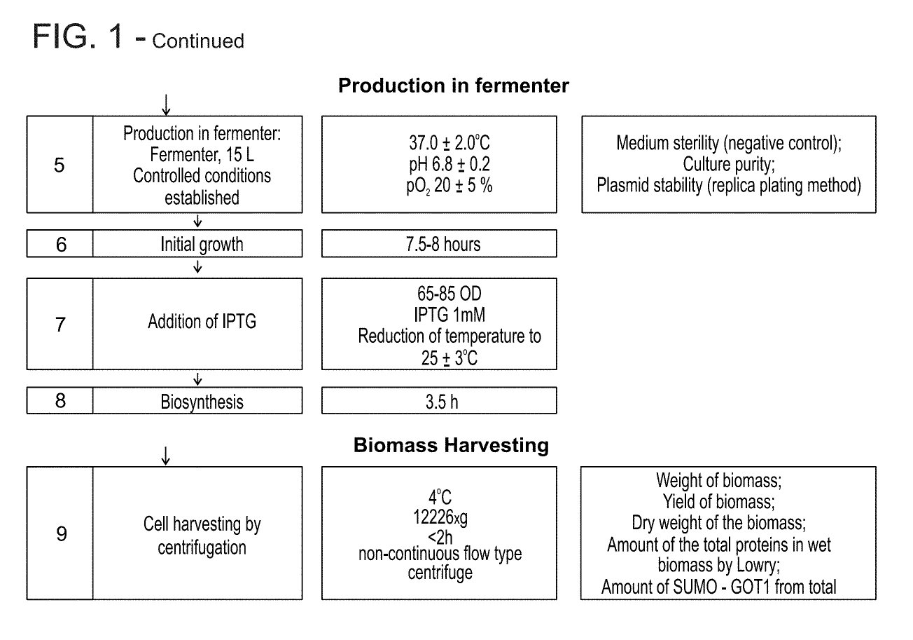

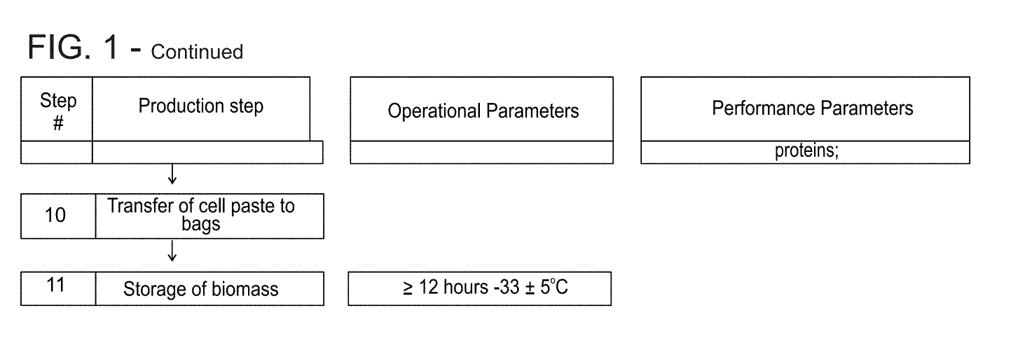

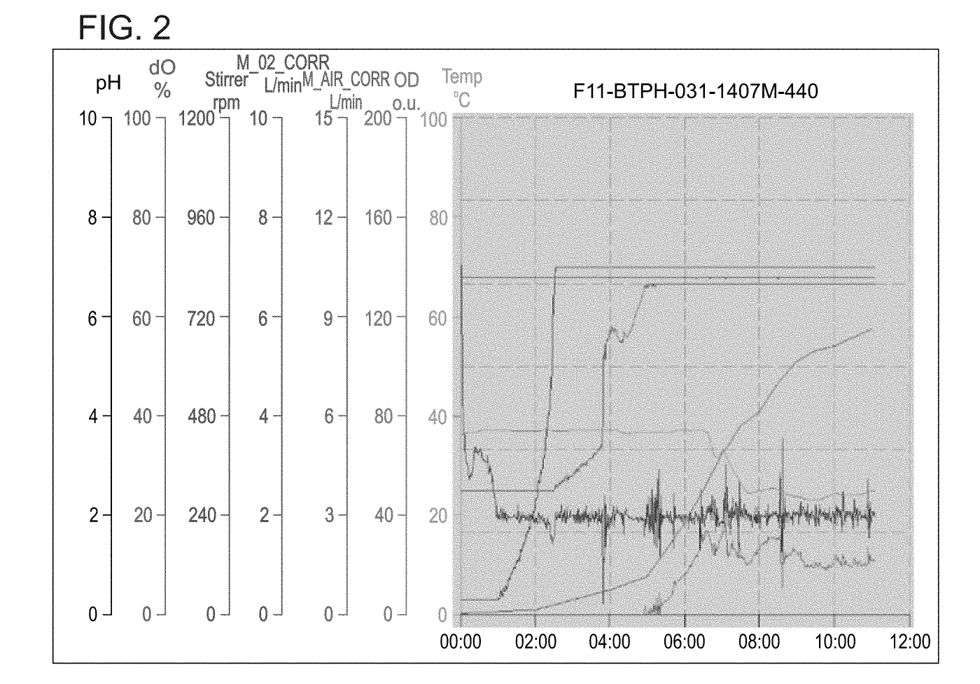

FIG. 1 shows the full scheme of the SUMO-GOT biosynthesis process.

Inoculum Preparation: An Erlenmeyer flask, containing 500 mL of sterile medium for cultivation in the flasks [a)], was inoculated with 0.20 mL of stock culture WCB (working cell bank) E. coli BL21 (DE3) pET28/SUMO-GOT1(Δhis). Thereafter, the flask was incubated in a rotating shaker with agitation speed 300 rpm, at 30° C. temperature for 18 hours. Optical density after incubation must be equal to or higher than 4.50 o.u. (optical units) [λ=595 nm].

Fermentation: The recombinant organism Escherichia coli SUMO-GOT1/ΔHis/pET28/E. coli BL21(DE3), from the research cell bank is grown for 17-19 hours in 1 L Erlenmeyer flask containing 0.5 L of growth medium. The flask was incubated in a shaker incubator at 30±2° C. and 300±50rpm shaking speed. Afterwards optical density of inoculum was measured at 600 nm wavelength and seeding volume is recalculated in order to obtain approximately 0.5 A.U. cell density in the fermenter (12 L working volume (15 L total volume) Applikon fermenter). A sample for culture purity was taken and tested by plating on nutrient media.

7.7 L of fermentation media was prepared and autoclaved together with 15 L fermenter. 700 mL of glucose and phosphate solutions were prepared and autoclaved separately and were transferred to the fermenter aseptically before fermentation. Base solution for pH maintenance and antifoam solution for foam control were also prepared separately and connected to the fermenter system before biosynthesis. The phosphate feeding solution and glucose feeding solution were prepared and autoclaved prior to biosynthesis. IPTG solution for induction of SUMO-GOT1 cells during biosynthesis was prepared and sterilized using sterile 0.2 μm filter shortly prior to usage. Before seeding the calculated volume of inoculum pH, temperature, agitation and dissolved oxygen control were established.

Set points of fermentation parameters were: 37° C. (up to the induction point) and 25° C. (after induction point) temperature that is ensured by controlled water flow in jacketed fermenter; 6.8 pH adjusted with ammonia solution; 20% O₂ concentration maintained with air and oxygen gas flows. The culture was grown at 37° C. until cell optical density reached 65-85 A.U. where sterile IPTG solution was added. After induction the culture was grown for 3.5 hours at the new temperature. Concentration of glucose (maintained at 17.0 g/L, concentration at feeding point) was measured every 30 min after cell density reached 20 A.U. and every 15 min after cell density reached 60-70 A.U. Carbon feed was performed according to measured glucose concentration in growth media. Four aliquots of phosphate feed solution were added during fermentation. The first was added when cell density reached 60-70 A.U and the rest were added according to time: 1 hour, 1.5 hour and 2 hours after induction. After fermentation the biomass was harvested and centrifugation performed. Immediately after fermentation culture purity testing was performed by plating samples on nutrient media.

The biomass from the fermenter was transferred to centrifugation bottles and centrifuged at 12227×g, 20 minutes at 4 DEG C. The biomass was then removed from the centrifuge bowls and transferred to plastic bags for further storage. The bags were placed in refrigerator at −33° C.

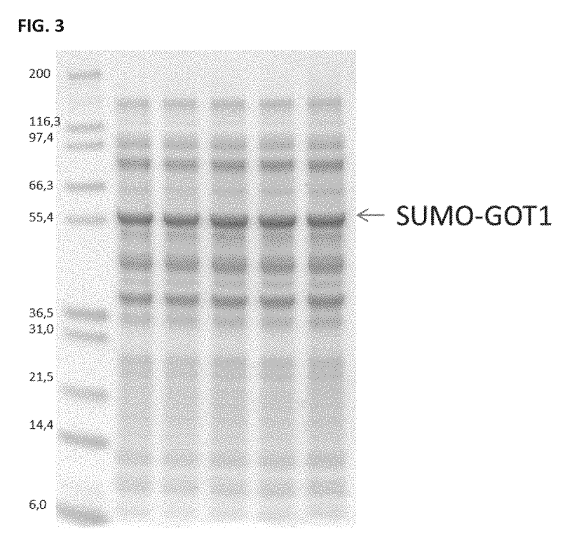

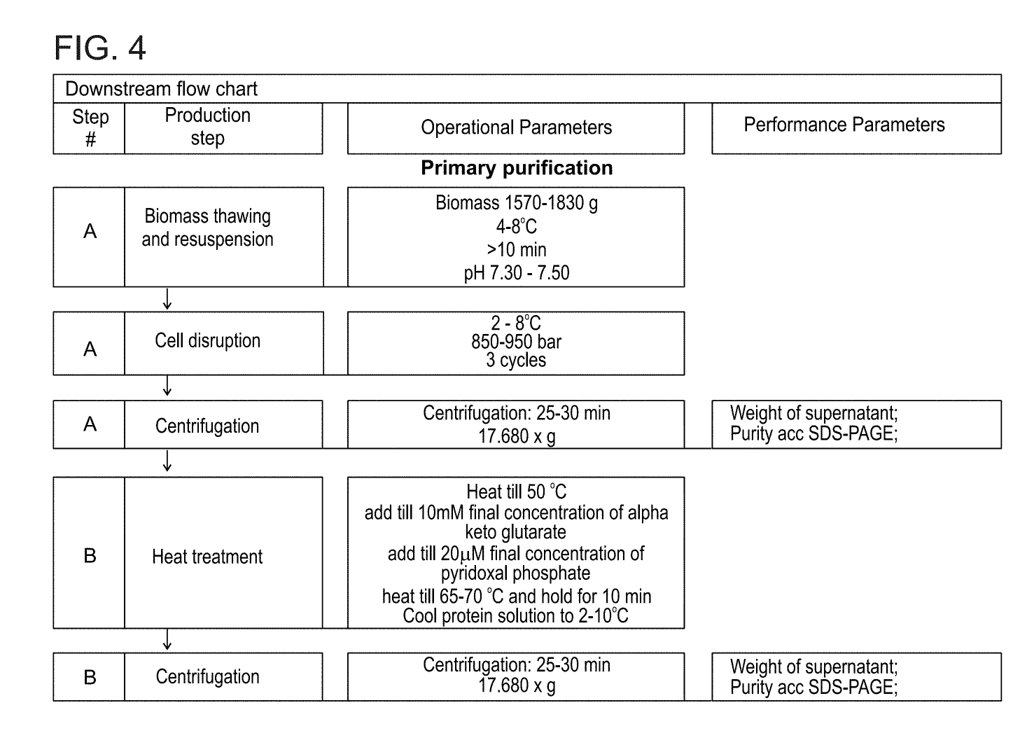

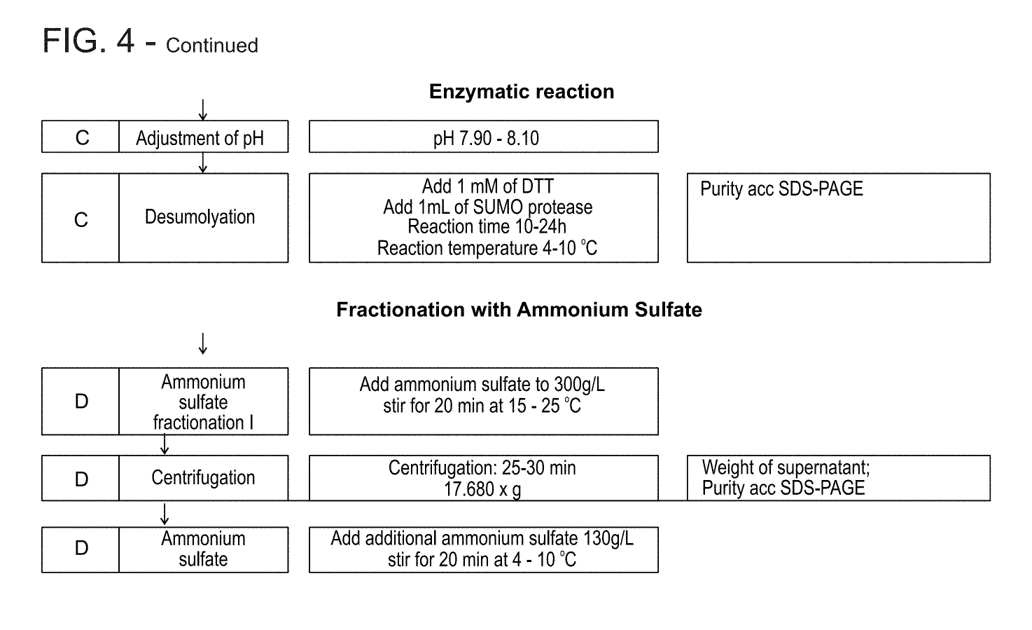

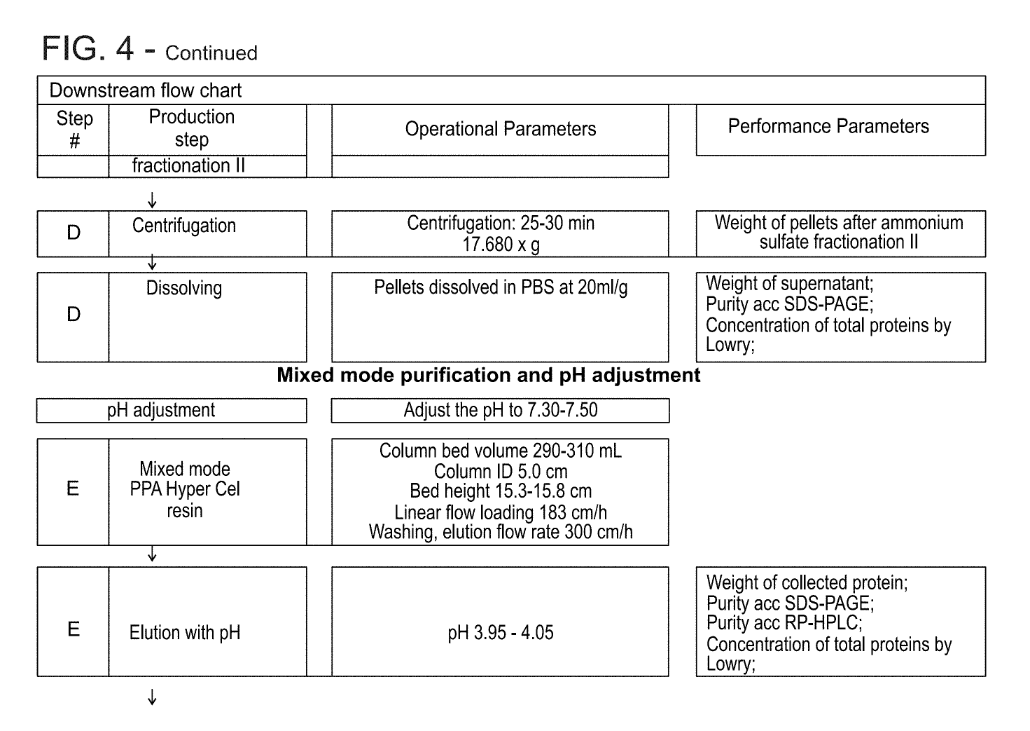

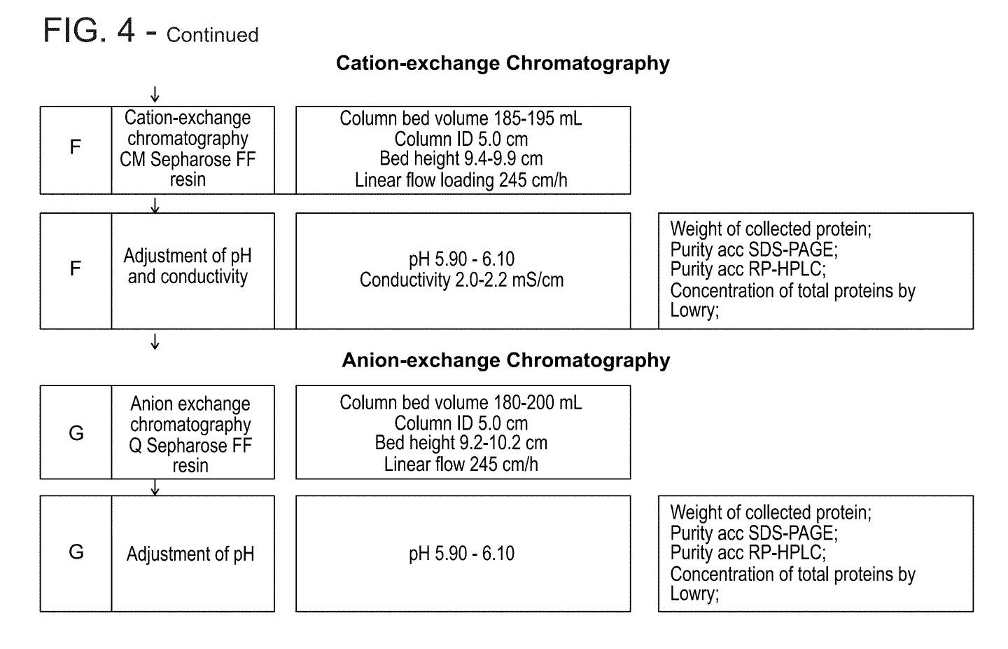

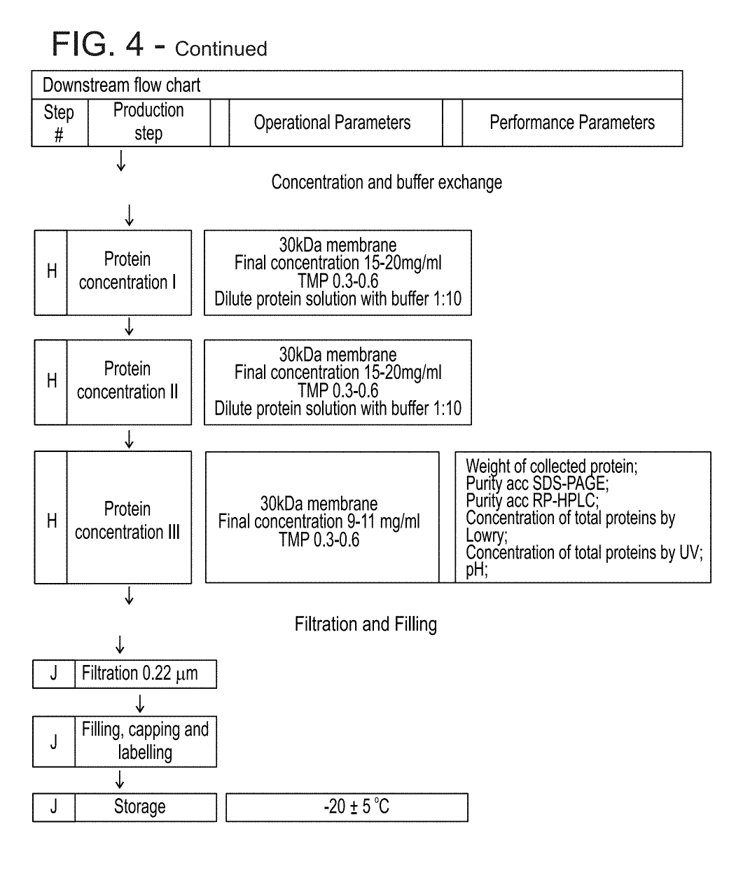

The sample was taken not earlier then after 12 hours of freeze storage and tested for total concentration of proteins (Lowry), SDS-PAGE analysis and gel scanning for SUMO-GOT1 quantity (FIG. 3).

### Example 2

### Description of the Primary Purification Process

Frozen pieces of the biomass (1570-1830 g) were removed from bags. The cells were reconstituted in a vessel with buffer solution W-A02 3 ml/gram of cell paste at temperature 4-8° C. The cells were disrupted using high pressure homogenizer at 900 bar pressure 3 cycles. The suspension was centrifuged, precipitate was discarded and protein solution was used for the next steps. The suspension was heated with stirring in the water bath and when temperature reached 50° C., alfa-ketoglutarate and pyridoxal 5-phosphate hydrate were added to a final concentration of 10 mM (an alfa-ketoglutarate) and 20 μM (pyridoxal 5-phosphate). The temperature was increased to 65-70° C. and the solution was incubated for 10±1 min. After cooling to 2-10° C., the denatured material was removed by centrifugation. The supernatant was decanted and collected. 1 mM of DTT was added to the protein solution after the heating treatment. 100,000U of SUMO protease was added and the temperature for cleavage was kept at 4-10° C. for 10-24 h. After deSUMOylation, solid ammonium sulphate was added to the protein solution, to a concentration of 300 g/L. This solution was stirred at room temperature for 20 min and centrifuged. The pellets were discarded and an additional amount of ammonium sulphate (130 g/L) was added to the supernatant fluid under continuous stirring. After 20 min storage at 4-10° C., the final pellets were collected by centrifugation and dissolved in PBS buffer 20 ml/g of pellets.

### Example 3

### Description of the Purification Process

Three chromatographic steps were utilized to purify the rGOT1 intermediate material: Mixed-mode interaction using medium PPA Hyper Cel Resin (bed height 15.3-15.8 cm), Ion-Exchange chromatography's using CM-Sepharose FF (bed height 9.4-9.9 cm) and Q Sepharose FF (bed height 9.2-10.2 cm).

Mixed-mode—PPA Hyper Cel: After pellets were dissolved, PPA Hyper Cel chromatography was performed. The pH of the protein solution was adjusted to 7.30-7.50, loaded and then eluted with a low pH buffer solution (pH 3.95-4.05) directly into the vessel. Fractions with optical density between 80 mAU (up) and 45 mAU (down) were collected (FIG. 5).

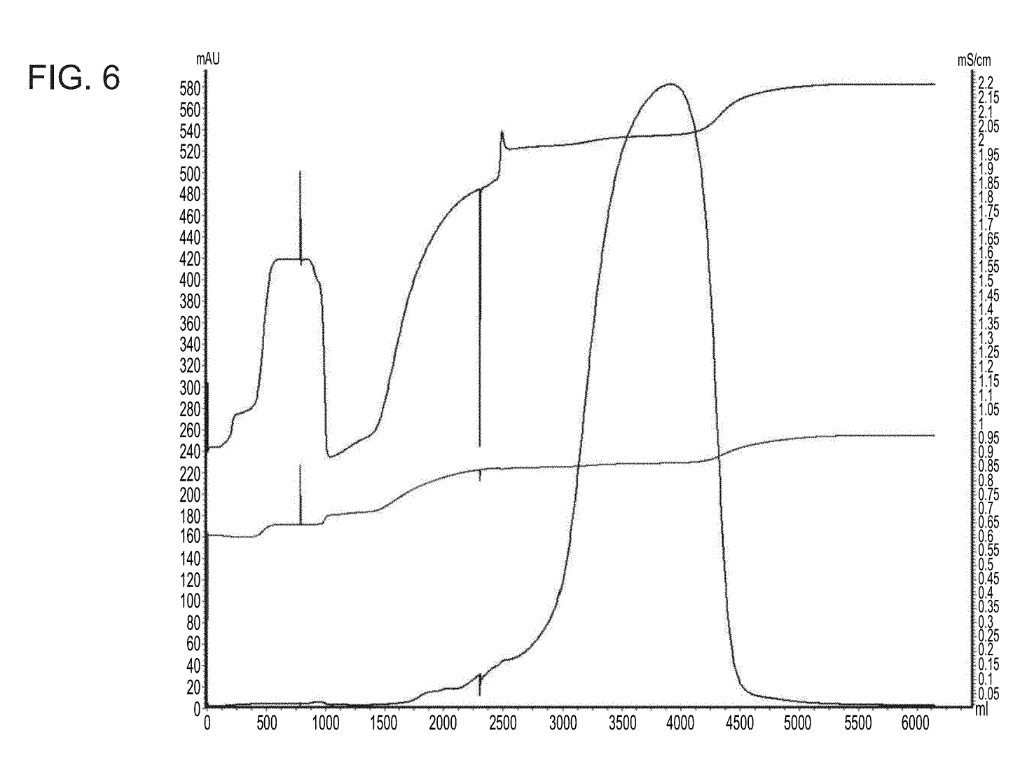

Cation exchange chromatography CM-sepharose FF: The eluted protein after Mixed-mode chromatography was loaded and then eluted with an increasing pH (W-E02) into a clean vessel. Protein fraction was collected from 80 mAU (up) till 35 mAU (down), the pH was adjusted to 5.90-6.10 and conductivity to 2.0-2.2 mS/cm (FIG. 6).

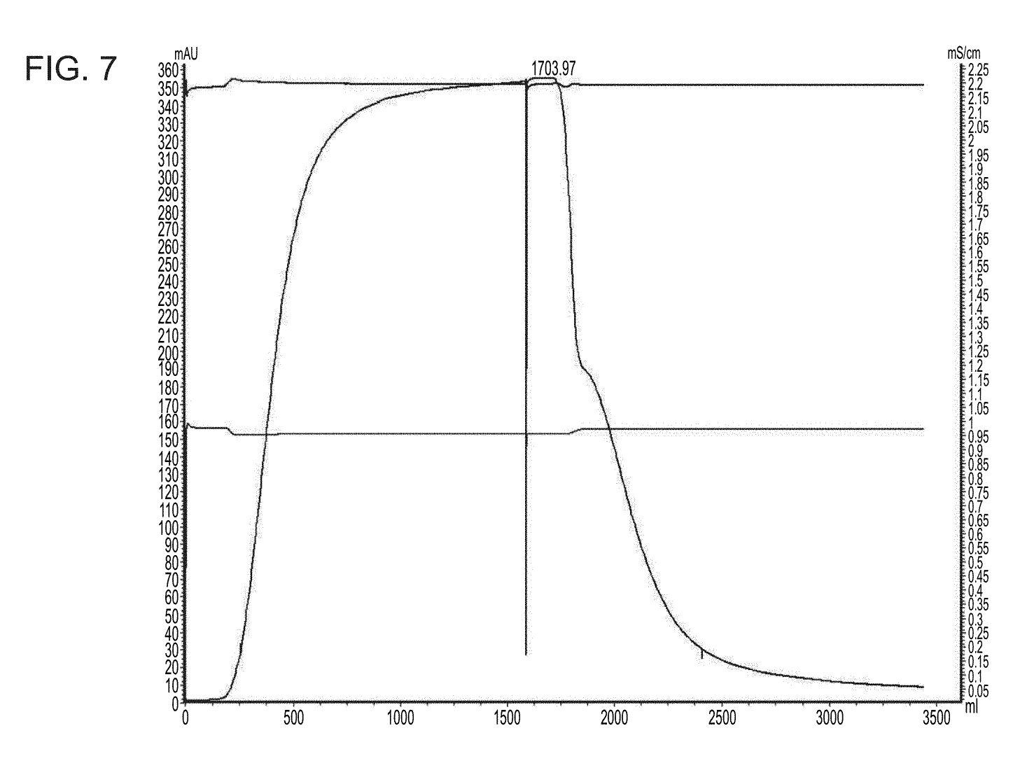

Anion chromatography Q Sepharose FF: The protein fraction was diluted until conductivity <2 mS/cm, pH was adjusted to pH 6.00 and loaded on the column. Protein fraction in flow through was collected from 35 mAU (up) till 30 mAU (down; FIG. 7).

### Example 4

### Formulation

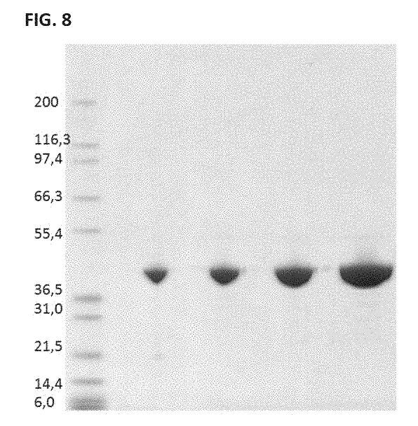

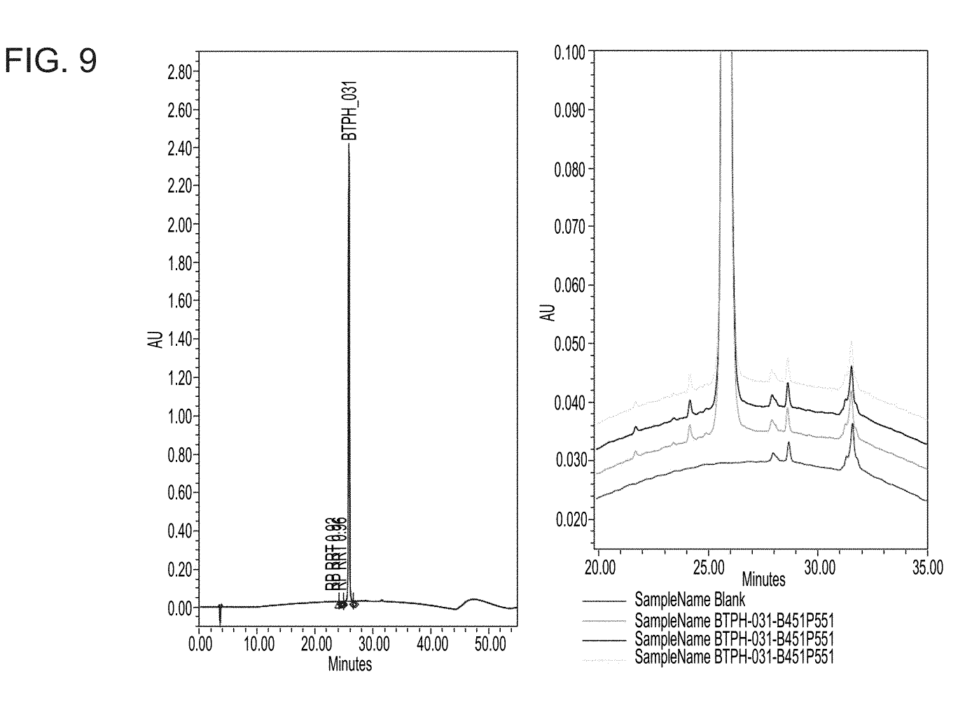

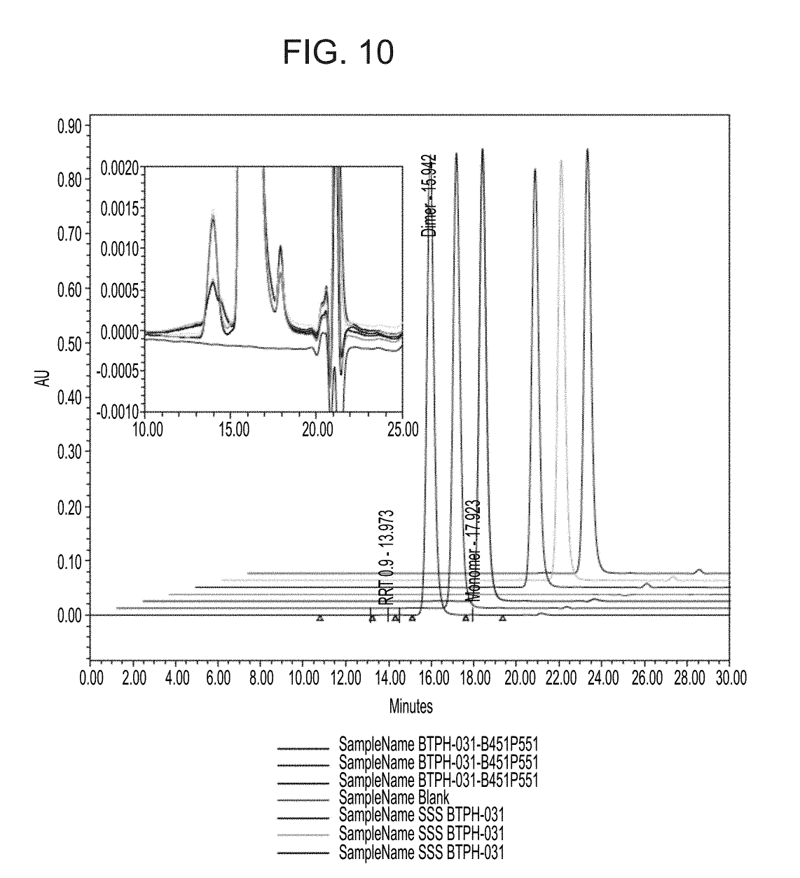

The protein solution from the anion exchange chromatography was concentrated using 30 kDa membrane to 15-20 mg/ml. 100 diafiltration volumes was used for buffer exchange to 20 mM Sodium acetate pH 5.0. Final protein concentration was 10±2 mg/ml. Transmembrane pressure was from 0.3 to 0.6. The purity was found to be >99% and the formulated GOT was analyzed by SDS-PAGE both under reducing conditions and non-reducing conditions (FIG. 8), RP-HPLC (FIG. 9), and SE-HPLC (FIG. 10). The results of the RP-HPLC analysis are summarized in Table 1, herein below.

TABLE 1

| | | RT | | % | | Symmetric | EP Plate | GOT1 |
|---|---|---|---|---|---|---|---|---|
| | Name | (min) | Area | Area | SN | Factor | Count | Related_proteins |
| 1 | BTPH_031 | 25.8 | 32152674 | 99.79 | 825.5 | 1 | 84949 | 0.21 |
| 2 | BTPH_031 | 25.8 | 31634473 | 99.77 | 825.6 | 1 | 86047 | 0.23 |
| 3 | BTPH_031 | 25.8 | 32296117 | 99.77 | 825.9 | 1 | 83607 | 0.23 |
| Std. | | 0 | 348061.4 | 0.01 | | | | 0 |
| Dev. | | | | | | | | |
| Mean | | 25.8 | 32027754 | 99.78 | 825.7 | 1 | 84867.8 | 0.2 |
| % | | 0 | 1.1 | 0.01 | | | | 5.5 |
| RSD | | | | | | | | |

The results of the SE-HPLC analysis are summarized in Tables 2-5, herein below.

TABLE 2
Dimer

| | | RT | | % | | | Symmetric | EP Plate | Channel |
|---|---|---|---|---|---|---|---|---|---|
| | Name | (min) | Area | Area | RT_RATIO | SN | Factor | Count | Description |
| 1 | Dimer | 15.9 | 19976316 | 99.66 | 1 | 191948.7 | 1.2 | 10856 | W2489 ChA |
| | | | | | | | | | 214 nm |
| 2 | Dimer | 15.9 | 20060097 | 99.64 | 1 | 182129.7 | 1.2 | 10738 | W2489 ChA |
| | | | | | | | | | 214 nm |
| 3 | Dimer | 15.9 | 20096245 | 99.66 | 1 | 164190.7 | 1.2 | 10859 | W2489 ChA |
| | | | | | | | | | 214 nm |
| Std. | | 0.0 | 61520.6 | 0.01 | 0 | | | | |
| Dev. | | | | | | | | | |
| Mean | | 15.9 | 20044219.5 | 99.65 | 1 | 179423 | 1.2 | 10817.9 | |
| % | | 0.0 | 0.3 | 0.01 | 0 | | | | |
| RSD | | | | | | | | | |

TABLE 3
Name: Monomer

| | | RT | | % | | | Channel |
|---|---|---|---|---|---|---|---|
| | Name | (min) | Area | Area | RT_RATIO | SN | Description |
| 1 | Monomer | 17.9 | 27892 | 0.14 | 1.124 | 237.6 | W2489 ChA |
| | | | | | | | 214 nm |
| 2 | Monomer | 17.9 | 27304 | 0.14 | 1.124 | 224.2 | W2489 ChA |
| | | | | | | | 214 nm |
| 3 | Monomer | 17.9 | 27665 | 0.14 | 1.124 | 202.4 | W2489 ChA |
| | | | | | | | 214 nm |
| Std. Dev. | | 0 | 296.5 | 0 | 0 | | |
| Mean | | 17.9 | 27620.2 | 0.14 | 1.124 | 221.4 | |
| % RSD | | 0 | 1.1 | 1.28 | 0 | | |

TABLE 4
RRT 0.9

| | | RT | | % | | | Channel |
|---|---|---|---|---|---|---|---|
| | Name | (min) | Area | Area | RT_RATIO | SN | Description |
| 1 | RRT | 14 | 24818 | 0.12 | 0.877 | 141.4 | W2489 ChA 214 nm |
| | 0.9 | | | | | | |
| 2 | RRT | 14 | 24465 | 0.12 | 0.877 | 119.1 | W2489 ChA 214 nm |
| | 0.9 | | | | | | |
| 3 | RRT | 14 | 25841 | 0.13 | 0.876 | 137.9 | W2489 ChA 214 nm |
| | 0.9 | | | | | | |
| Std. | | 0 | 714.4 | 0 | 0 | | |
| Dev. | | | | | | | |
| Mean | | 14 | 25041.2 | 0.12 | 0.877 | 132.8 | |
| % RSD | | 0 | 2.9 | 2.86 | 0.015 | | |

TABLE 5
Name: Total HMW

| | | | | | | | Channel |
|---|---|---|---|---|---|---|---|
| | Name | RT (min) | Area | % Area | RT_RATIO | SN | Description |
| 1 | Total | 13.1 | 45055 | 0.22 | 0.823 | 246.7 | W2489 ChA |
| | HMW | | | | | | 214 nm |
| 2 | Total | 13.1 | 40421 | 0.2 | 0.822 | 207.2 | W2489 ChA |
| | HMW | | | | | | 214 nm |
| 3 | Total | 12.5 | 41077 | 0.2 | 0.783 | 261.1 | W2489 ChA |
| | HMW | | | | | | 214 nm |
| Std. Dev | | 0 | 2507.5 | 0.01 | 0.023 | | |
| Mean | | 12.9 | 42184.3 | 0.21 | 0.809 | 238.3 | |
| % RSD | | 0 | 5.9 | 5.91 | 2.817 | | |

The results of the peptide mapping are illustrated in FIG. 11. The results of the isoelectric focusing studies are illustrated in FIG. 12. Balance of purification is presented in Table 6, herein below.

TABLE 6

| | | | | | | | | | | | | Purity acc | |
|---|---|---|---|---|---|---|---|---|---|---|---|---|---|
| | Concen- | | | | | Total | | | Specific | Purification | Puri- | SDS- | Purity acc, |
| | tration | Volume, | Quantity, | Step, | Total, | activity, | Step, | Total, | activity, | fold per | fication | PAGE, | RP-HPLC, |
| STEP | mg/mL | ml | mg | % | % | U | % | % | U/mg | step | factor | % | % |
| After disruption | 62.9 | 4530 | 284691 | 100 | 100.0 | 1810374 | 100 | 100 | 6 | 1 | 1 | 24 | — |
| After heating | 14.7 | 3559 | 52389 | 18 | 18.4 | 1647259 | 91 | 91 | 31 | 4.9 | 5 | 63 | — |
| treatment | | | | | | | | | | | | | |
| After | 14.6 | 3559 | 51878 | 99 | 18.2 | 1458489 | 89 | 81 | 28 | 0.9 | 4 | 42 | — |
| deSUMOlyation | | | | | | | | | | | | | |
| After AmSO4 | 4.9 | 4440 | 21762 | 42 | 7.6 | 1227998 | 84 | 68 | 56 | 2.0 | 9 | 67 | — |
| frac I | | | | | | | | | | | | | |
| Pellets | 4.9 | 1533 | 7533 | 35 | 2.6 | 981013 | 80 | 54 | 130 | 2.3 | 20 | 67 | — |
| dissolving | | | | | | | | | | | | | |
| After PPA | 3.2 | 1040 | 3306 | 44 | 1.2 | — | — | — | — | — | — | 82 | 91.9 |
| After CM | 1.6 | 1504 | 2476 | 75 | 0.9 | 402451 | 100 | 22 | 163 | — | 26 | 86 | 94.7 |
| After Q | 1.0 | 2094 | 2120 | 86 | 0.7 | 404518 | 100 | 22 | 191 | 1.2 | 30 | 99 | 99.4 |
| After TFF | 12.4 | 132 | 1633 | 77 | 0.6 | 413397 | 100 | 22 | 253 | 1.3 | 40 | 99 | 99.8 |

The specific activity of the enzyme, measured using Karmen method was shown to be 200 U/mg.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A protein preparation comprising Glutamate Oxaloacetate Transaminase 1 (GOT1) polypeptide molecules, wherein 100% of the GOT1 polypeptide molecules have an alanine at position 1 of the GOT1 polypeptide, and wherein the GOT1 polypeptide molecules constitute at least 95% of the proteins in the preparation.

2. The protein preparation of claim 1, wherein the protein constitutes at least 98% of the molecules in the preparation.

3. (canceled)

4. The preparation of claim 1, wherein said GOT1 comprises an amino acid sequence at least 90% homologous to SEQ ID NO: 2.

5. The preparation of claim 1, wherein said GOT1 consists of the amino acid sequence as set forth in SEQ ID NO: 2.

6. (canceled)

7. A Glutamate Oxaloacetate Transaminase 1 (GOT1) fusion protein comprising GOT1 and Small Ubiquitin-like Modifier (SUMO), wherein the N terminal of said GOT1 is translationally fused to the C terminal of said SUMO, wherein the fusion protein is devoid of an affinity tag.

8. The fusion protein of claim 7, wherein said GOT1 comprises an alanine at position 1.

9-12. (canceled)

13. The fusion protein of claim 7, consisting of the amino acid sequence as set forth in SEQ ID NO: 1.

14. The fusion protein of claim 7, wherein said SUMO is a yeast SUMO.

15. (canceled)

16. An isolated polynucleotide encoding the fusion protein of claim 7, wherein said polynucleotide is devoid of a sequence that encodes an affinity tag.

17. The isolated polynucleotide of claim 16, comprising a nucleic acid sequence at least 90% homologous to SEQ ID NO: 3.

18. A nucleic acid construct comprising the isolated polynucleotide of claim 16.

19. A method of treating a disease or condition associated with an excess of glutamate in a subject in need thereof, comprising administering to the subject a therapeutically effective amount of the preparation of claim 1, thereby treating the disease or condition.

20-21. (canceled)

22. The method of claim 19, wherein the disease or condition is a brain disease or condition.

23. The method of claim 22, wherein said brain disease is a cancer of the central nervous system or a neurodegenerative disease.

24. The method of claim 23, wherein said cancer is a glioblastoma.

25. The method of claim 22 wherein said brain condition is cerebral ischemia.

26. (canceled)

27. A method of purifying a polypeptide comprising:
(a) expressing a fusion protein comprising said polypeptide and SUMO in host cells, wherein the N terminal of said polypeptide is translationally fused to the C terminal of said SUMO, wherein the fusion protein is devoid of a heterologous affinity tag;
(b) removing said SUMO from said fusion protein; and
(c) isolating said polypeptide from said host cells, thereby purifying the polypeptide.

28. (canceled)

29. The method of claim 27, wherein said polypeptide is GOT1.

30-32. (canceled)

33. The method of claim 29, wherein said isolating is effected by:
(a) purifying the GOT1 by heat treatment;
(b) purifying the GOT1 by salt induced precipitation;
(c) purifying the GOT1 by mixed mode chromatography;
(d) purifying the GOT1 by cation exchange chromatography; and
(e) purifying the GOT1 by anion exchange chromatography, wherein step (e) follows step (d), wherein step (d) follows step (c), wherein step (c) follows step (b) and wherein step (b) follows step (a).

34-36. (canceled)
